(19) United States
(12) Patent Application Publication
(10) Pub. No.: US 2017/0065763 A1
(43) Pub. Date: Mar. 9, 2017

(54) Universal Adapter for a Medical Injector and Syringe Identification System

(71) Applicant: BAYER HEALTHCARE LLC, Whippany, NJ (US)

(72) Inventors: Vincent S. Rossitto, Apollo, PA (US); Christopher D. Capone, Pittsburgh, PA (US); Alexander Flamm, Baltimore, MD (US); Andrew Rogers, Baltimore, MD (US); Keith Lipford, Baltimore, MD (US); Chet Larrow, Baltimore, MD (US); Frank Regan, Baltimore, MD (US)

(21) Appl. No.: 15/121,325

(22) PCT Filed: Feb. 26, 2015
(86) PCT No.: PCT/US15/17650

§ 371 Date: Aug. 24, 2016

Related U.S. Application Data
(60) Provisional application No. 61/946,421, filed on Feb. 28, 2014.

(51) Int. Cl. A61M 5/00; A61M 5/315; A61M 5/19; A61M 5/31; A61M 5/145; A61M 5/168

(52) U.S. Cl. CPC A61M 5/007; A61M 5/14546; A61M 5/1684; A61M 5/19; A61M 5/3134; A61M 5/31515; A61M 2209/084; A61M 2205/332; A61M 2205/3389; A61M 2205/6072; A61M 2205/3334

(57) ABSTRACT

A universal adapter for connecting a syringe to an injector is provided. The universal adapter for connecting a syringe to an injector includes: a body having a proximal end configured to connect to an injector; at least one radial support connected to the body and biased in an inward direction; and at least one axial support biased to position the syringe in an axial direction toward a distal end of the adapter. The at least one radial support defines a notch positioned to contact and engage a portion of a barrel of the syringe. A fluid delivery system and system for identification of a syringe, which include a universal adapter for connecting a syringe to an injector, are also described herein.

### CROSS REFERENCE TO RELATED APPLICATION

The present application claims priority to U.S. Provisional Application No. 61/946,421 filed Feb. 28, 2014, the disclosure of which is incorporated by reference herein.

### BACKGROUND

Field

This disclosure relates, in general, to the field of medical injectors, and, more particularly, to a universal adapter for a medical injector, as well as a system for identifying the physical dimensions and other physical parameters of a syringe contained in the universal adapter.

Description of the Related Art

In many medical diagnostic and therapeutic procedures, a medical practitioner, such as a physician, injects a patient with a fluid. In recent years, a number of injector-actuated syringes and powered injectors for pressurized injection of fluids, such as contrast media (often referred to simply as “contrast”), have been developed for use in procedures such as angiography, computed tomography, ultrasound, and NMR/MRI. In general, these powered injectors are designed to deliver a preset amount of contrast at a preset flow rate and pressure.

Angiography is used in the detection and treatment of abnormalities or restrictions in blood vessels. In an angiographic procedure, a radiographic image of a vascular structure is obtained through the use of a radiographic contrast that is injected through a catheter. The vascular structures in fluid connection with the vein or artery in which the contrast is injected are filled with contrast. X-rays passing through the region of interest are absorbed by the contrast, causing a radiographic outline or image of blood vessels containing the contrast. The resulting images can be displayed on, for example, a video monitor and recorded.

In a typical angiographic procedure, the medical practitioner places a cardiac catheter into a vein or artery. The catheter is connected to either a manual or an automatic contrast injection mechanism. A typical manual contrast injection mechanism includes a syringe in fluid connection with a catheter connection. The fluid path also includes, for example, a source of contrast and a source of flushing fluid, typically saline. The operator of the manual contrast injection mechanism controls the syringe to draw saline or contrast into the syringe and to inject the contrast or saline into a patient through the catheter connection.

Automatic contrast injection mechanisms typically include a syringe connected to a powered injector having, for example, a powered linear actuator. The linear actuator operates a plunger rod configured to contact and engage a moveable plunger of the syringe. Typically, an adaptor structure is used to precisely mount the syringe in line with the linear actuator of the injector, in a manner in which the fluid content of the syringe can be accurately dispensed under flow rate, volume, and pressure controls. In currently available fluid delivery systems, an operator selects a specific adapter from among alternative adapters for the syringe being used for a particular procedure. If a different sized syringe is needed for a later procedure, the operator must remove and replace the adapter with a new adapter sized for the new syringe. The process of exchanging adapters reduces efficiency and increases time required for certain injection procedures.

Once the syringe is inserted in the correct sized adapter, the operator enters settings into an electronic control system of the powered injector that control fluid delivery pressure and volume. In some systems, there is no interactive control between the operator and the powered injector, except to start or stop the injection. A change in flow rate in such systems occurs by stopping the machine and manually resetting the injection parameters. Automated systems for controlling powered injectors are also known. Automation of angiographic procedures using powered injectors is discussed, for example, in U.S. Pat. Nos. 6,339,718; 6,397,098; and 6,643,537, assigned to the assignee of the present application. However, such automated systems may still require a user or operator to identify the type of syringe connected to the powered injector. Syringe identification is required to accurately convert linear piston travel of the linear actuator and resulting forces to fluid delivery parameters, such as fluid volume and pressure. Such syringe identification is generally necessary to support safe and accurate control of contrast fluid delivery from prefilled syringes that have different geometries and physical dimensions, different barrel/plunger characteristics, different material properties and structural strengths, and different pressure limitations. Accordingly, even automated powered injectors require significant input and information from the operator.

In view of the difficulties in configuring a powered injector for different sized syringes, there is a need for an adapter that can be used with different geometries and types of syringes. Furthermore, there is a need for integration between the adapter and electronic control system of the injector so that the injector settings can be easily and automatically adjusted for each new syringe type. The system should identify the type of syringe being used and should use that information to make appropriate changes to the injector settings, as needed. The universal adapter and syringe identification system provided herein are configured to address these issues.

### SUMMARY

According to an aspect of the disclosure, a universal adapter for connecting a syringe to an injector includes: a body having a proximal end configured to connect to an injector; at least one radial support connected to the body and biased in an inward direction; and at least one axial support biased to position the syringe in an axial direction toward a distal end of the adapter. The at least one radial support defines a notch positioned to contact and engage a portion of a barrel of the syringe.

According to another aspect of the disclosure, a universal adapter for connecting a syringe to an injector includes a housing having a proximal end configured to connect to an injector. The housing defines a cavity configured to receive a syringe. The adapter also includes at least one radial support positioned in the cavity and biased in an inward direction. The at least one radial support is configured to substantially align a longitudinal axis of the syringe with a longitudinal axis of the housing. The adapter can also include at least one axial support at least partially positioned within the cavity and biased to position the syringe toward a distal end of the adapter. The radial support and the axial support are configured to receive and to provide alignment for syringes across a range of different dimensions and geometries.

According to another aspect of the disclosure, a fluid delivery system includes: a syringe for delivering a fluid to a patient, the syringe comprising a barrel and a plunger slidably disposed within the barrel; an injector comprising a linear actuator; and a universal adapter configured to receive the syringe and to align the syringe with the linear actuator of the injector. Once connected together, the linear actuator is configured to advance the plunger through the barrel to expel fluid from the syringe. The universal adapter includes: a body having a proximal end configured to connect to the injector; at least one radial support connected to the body and biased in an inward direction; and at least one axial support biased to position the syringe in an axial direction toward a distal end of the adapter. The at least one radial support defining a notch positioned to contact and engage the syringe barrel.

According to another aspect of the disclosure, a fluid delivery system includes: a syringe for delivering a fluid to a patient having a barrel and a plunger slidably disposed within the barrel; an injector comprising a linear actuator; and a universal adapter configured to receive the syringe and to align the syringe with the linear actuator of the injector, such that the linear actuator can advance the plunger through the barrel to expel fluid from the syringe. The universal adapter includes: a housing having a proximal end configured to connect to the linear actuator of the injector and defining a cavity configured to receive the syringe; at least one radial support positioned in the cavity and biased in an inward direction, the at least one radial support being configured to substantially align a longitudinal axis of the syringe with a longitudinal axis of the housing; and at least one axial support at least partially positioned within the cavity and being biased to position the syringe toward a distal end of the adapter. The radial support and the axial support are configured to receive and to provide alignment for syringes across a range of different dimensions and geometries.

According to another aspect of the disclosure, a syringe identification system includes: at least one syringe containing a medical fluid for injection to a patient; an injector comprising a linear actuator configured to expel fluid from the syringe; and a universal adapter for receiving the syringe and for aligning the syringe with the linear actuator of the injector. The universal adapter includes: a body having a proximal end configured to connect to the injector; at least one radial support connected to the body and biased in an inward direction; and at least one axial support biased to position the syringe in an axial direction toward a distal end of the adapter. The at least one radial support defines a notch positioned to contact and engage a portion of a barrel of the syringe. The syringe identification system also includes one or more sensors disposed on or associated with the universal adapter or the injector. The sensors are configured to obtain measurements for dimensions and geometries of the syringe. The measurements obtained by the one or more sensors are used to identify a type of syringe, a syringe fluid volume, syringe fluid flow characteristics, or any combination thereof.

According to another aspect of the disclosure, a syringe identification system includes: at least one syringe containing a medical fluid for injection to a patient; an injector comprising a linear actuator configured to expel fluid from the syringe; and a universal adapter for receiving the syringe and for aligning the syringe with the linear actuator of the injector. The universal adapter is configured to receive and to provide alignment for syringes across a range of different dimensions and geometries. The syringe identification system can also include one or more sensors disposed on or associated with the universal adapter or the injector. The sensors are configured to obtain measurements for dimensions and geometries of the syringe. The measurements obtained by the one or more sensors can be used to identify a type of syringe, a syringe fluid volume, syringe fluid flow characteristics, or any combination thereof.

These and other features and characteristics of the universal adapter and syringe identification system, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limits of the disclosure. As used in the specification and the claims, the singular form of “a”, “an”, and “the” include plural referents unless the context clearly dictates otherwise.

### BRIEF DESCRIPTION OF THE DRAWINGS

FIG. 1 is a perspective view an embodiment of a powered injector;

FIG. 2A is a perspective view of an embodiment of a universal adapter, for use with a powered injector;

FIG. 2B is a schematic drawing of a support structure of the universal adapter of FIG. 2A;

FIG. 2C is a schematic drawing of a plunger rod and spring of the universal adapter of FIG. 2A;

FIGS. 3A-3C are schematic drawings depicting the steps of inserting a pre-filled syringe into the universal adapter of FIG. 2A;

FIG. 4A is a perspective view of another embodiment of a universal adapter;

FIG. 4B is a schematic drawing of a support structure of the universal adapter of FIG. 4A;

FIG. 4C is a schematic drawing of a plunger rod and spring of the universal adapter of FIG. 4A;

FIG. 5A is a perspective view of another embodiment of a universal adapter;

FIG. 5B is a schematic drawing of a support structure of the universal adapter of FIG. 5A;

FIG. 5C is a schematic drawing of springs and a plunger rod of the universal adapter of FIG. 5A;

FIG. 6A is a perspective view of an embodiment of a fluid injector and universal adapter;

FIG. 6B is a front perspective view of the fluid injector and universal adapter of FIG. 6A;

FIG. 7A is a perspective view of the universal adapter of FIG. 6A with a portion of the adapter housing removed therefrom;

FIG. 7B is a cross sectional view of the universal adapter of FIG. 7A;

FIGS. 8A-8E are schematic views illustrating insertion of a syringe into the universal adapter and fluid injector of FIG. 7A;

FIGS. 9A-9E are schematic drawings of the interface between the adapter and fluid injector of FIG. 7A;

FIGS. 10A-10D are schematic drawings of the biasing mechanism of the universal adapter of FIG. 7A;

FIGS. 11A-11C are perspective views of the adapter of FIG. 7A with a portion of the adapter housing removed therefrom;

FIG. 12A is a schematic drawing of the outer diameter detector of the adapter of FIG. 7A;

FIG. 12B is a perspective view of the interface between the adapter and injector of FIG. 7A;

FIGS. 13A and 13B are schematic views of an optical sensor for use with the adapter of FIG. 7A;

FIGS. 14A and 14B are perspective views of another embodiment of a fluid injector having two universal adapters;

FIGS. 15A-15D are perspective views of another embodiment of the universal adapter with a portion of the housing removed therefrom, illustrating advancement of the drive member assembly from an initial to a syringe zero volume position;

FIGS. 16A-16D are perspective views of the universal adapter of FIGS. 15-15B, when the syringe is in a retracted position;

FIG. 17A is a schematic drawing of an embodiment of a syringe identification and injection system;

FIGS. 17B and 17C are schematic drawings showing steps for using the system of FIG. 17A;

FIG. 18A is a schematic drawing of another embodiment of a syringe identification and injection system;

FIGS. 18B-18D are schematic drawings showing steps for using the system of FIG. 18A;

FIG. 19A is a schematic drawing of another embodiment of a syringe identification and injection system;

FIGS. 19B and 19C are schematic drawings showing steps for using the system of FIG. 19A;

FIGS. 20A and 20B are schematic drawings showing alternative steps for using the system of FIG. 19A;

FIG. 21 is a schematic drawing of another embodiment of a syringe identification and injection system;

FIG. 22A is a schematic drawing of another embodiment of a syringe identification and injection system for syringe identification;

FIGS. 22B and 22C are schematic drawings showing steps for using the system of FIG. 22A;

FIGS. 23A-23C are perspective views of an adapter according to a further embodiment with a portion of the adapter housing removed therefrom;

FIGS. 24A-24D are perspective views of portions of the adapter of FIGS. 23A-23C; and

FIGS. 25A and 25B are perspective views of additional portions of the adapter of FIGS. 23A-23C.

### DETAILED DESCRIPTION

For purposes of the description hereinafter, spatial orientation terms, if used, shall relate to the referenced embodiment as it is oriented in the accompanying drawing figures or otherwise described in the following detailed description. Particularly, the term “proximal” refers to an end of a syringe nearer to an operator's hand or to a drive mechanism of a powered injector. The term “distal” refers to the end of a syringe farthest away from the operator's hand, where fluid is ejected from the syringe. However, it is to be understood that the embodiments described hereinafter may assume many alternative variations and embodiments. It is also to be understood that the specific devices illustrated in the accompanying drawing figures and described herein are simply exemplary and should not be considered as limiting.

Referring to the drawings in which like reference characters refer to like parts throughout the several views thereof, an injector for injecting one or more medical fluids to a patient is illustrated in detail.

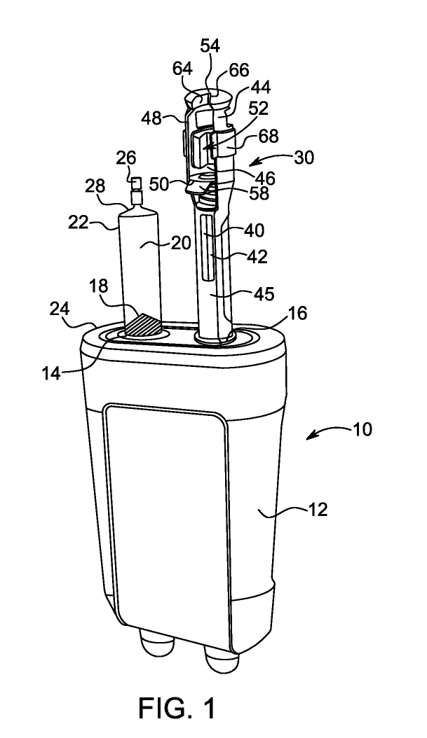

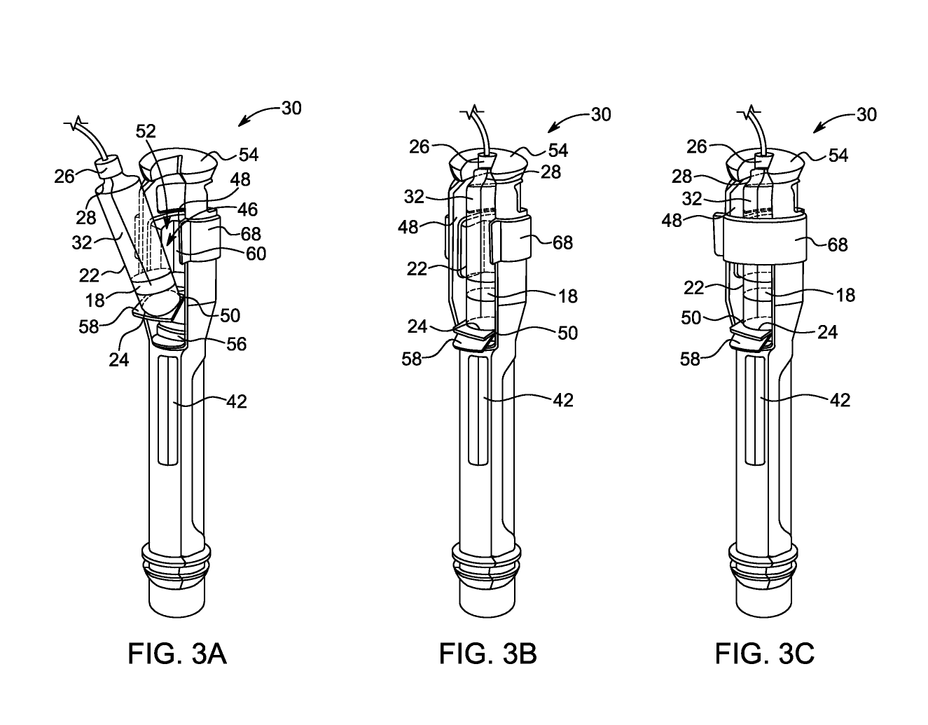

With reference to FIG. 1, an injector 10, such as an automatic or powered injector, is illustrated, which is adapted to interface with and actuate a plurality of syringes. The syringes may be filled with contrast media, saline solution, or any desired medical fluids. For example, a first syringe, referred to hereinafter as a saline syringe 20, may be filled with the saline solution. A second syringe, referred to hereinafter as a contrast syringe 32 (shown in FIG. 3A), may be filled with the contrast media. The powered injector 10 may be used during an angiographic procedure to inject contrast and common flushing agents, such as saline, into the body of a patient. The powered injector 10 is desirably at least a dual-syringe injector, wherein the two fluid delivery syringes are oriented in a side-by-side relationship and are separately actuated by respective linear actuators and/or piston elements, associated with the powered injector 10.

The injector 10 may be enclosed within a housing 12 formed from a suitable structural material such as medical grade plastic. The housing 12 may be in various shapes and sizes depending on the desired application. For example, the injector 10 may be a free-standing structure configured to be placed on the floor or may be a smaller design for placement on a suitable table or frame. The injector 10 includes syringe ports for connecting the saline syringe 20 and the contrast syringe 32 to respective linear actuators and/or piston elements. The syringe ports, referred to hereinafter as a first syringe port 14 and a second syringe port 16, are located on a top side of the housing 12. As shown in FIG. 1, the saline syringe 20 is connected directly to the first syringe port 14. The contrast syringe 32 is connected to the second syringe port 16 with a universal adapter 30 configured to hold syringes of various shapes and sizes.

The syringes 20, 32 generally have a cylindrical syringe barrel 22 formed from glass or medical-grade plastic. The barrel 22 has an open proximal end 24 and a nozzle 26 extending from its distal end 28. The open proximal end 24 may be sealed with an elastomeric plunger 18 that is capable of forming a fluid tight seal against a sidewall of the barrel 22. The plunger 18 is configured to slide through the syringe barrel 22.

A fluid path set (not shown in FIG. 1) may be interfaced with powered injector 10 for delivering fluid from the syringes 20, 32 to a catheter (not shown) for insertion into a patient at a vascular access site. The flow of saline solution from the saline syringe 20 and contrast from the contrast syringe 32 may be regulated by a fluid control module (not shown) which controls various valves and flow regulating structures to regulate the delivery of the saline solution and contrast to the patient based on user selected injection parameters, such as injection flow rate, duration, total injection volume, and ratio of contrast media and saline. A suitable multi-syringe injector 10 is described in U.S. patent application Ser. No. 13/386,765, filed on Jan. 24, 2012, published as U.S. Patent Application Publication No. 2012/0123257, and assigned to the assignee of the present application, the disclosure of which is incorporated herein by reference in its entirety. Other relevant multi-fluid delivery systems are found in U.S. patent application Ser. No. 10/159,592, filed on May 30, 2002 (published as U.S. 2004/0064041) and in U.S. patent application Ser. No. 10/722,370, filed Nov. 25, 2003 (published as U.S. 2005/0113754), assigned to the assignee of the present application, and the disclosures of which are both incorporated herein by reference.

With reference to FIGS. 1, 2A, and 3A, a preferred and non-limiting embodiment of the universal adapter 30 is illustrated. The universal adapter 30 is configured for physical mounting of prefilled syringes across a range of dimensions and geometries that fall within pre-designated constraints, to the injector 10. The universal adapter 30 includes a substantially tubular structure, housing, or body having a proximal end 34 configured to connect the contrast syringe 32 to the second syringe port 16 of the injector 10. The adapter 30 provides radial and axial restraint to ensure the syringe 32 is positioned properly during power injection. In addition, the adapter 30 provides forward bias of the syringe 32 within the adaptor 30 to minimize mechanical slack related errors and to provide an absolute positional reference for injector motion control. As will be described hereinafter, the adapter 30 may also provide detection and identification of the syringe 32 mounted in the adapter 30. The adapter 30 may also provide certain secondary identifications of the syringe 32 to further reduce the risk of an incorrect syringe 32 being mounted to the adapter 30.

The universal adapter 30 is generally divided into two parts, a proximal drive rod housing portion 45 and a distal syringe receiving portion 44. The portions 44, 45 of the adapter 30 (collecting referred to as the adapter housing or body) may be removable from one another or permanently connected together. The drive rod housing portion 45 encloses a moveable drive rod 42. To facilitate connection with the syringe port 16, the drive rod housing portion 45 may include one or more fastening structures, such as one or more annular flanges 36 extending about the proximal end 34 of the adapter 30. The proximal end 34 of the adapter 30 also includes an opening 38 or aperture for connecting the drive rod 42 to the linear actuator (not shown) of the injector 10. During an injection procedure, the linear actuator advances the drive rod 42 toward the proximal end of the plunger 18, causing the drive rod 42 to engage the plunger 18. For example, the drive rod 42 and/or plunger 18 may include corresponding locks or latching structures that fit together to form a removable engagement therebetween. The drive rod 42 is viewable through a window 40. An operator can determine the progress of the injection by viewing the position of the drive rod 42 through the window 40.

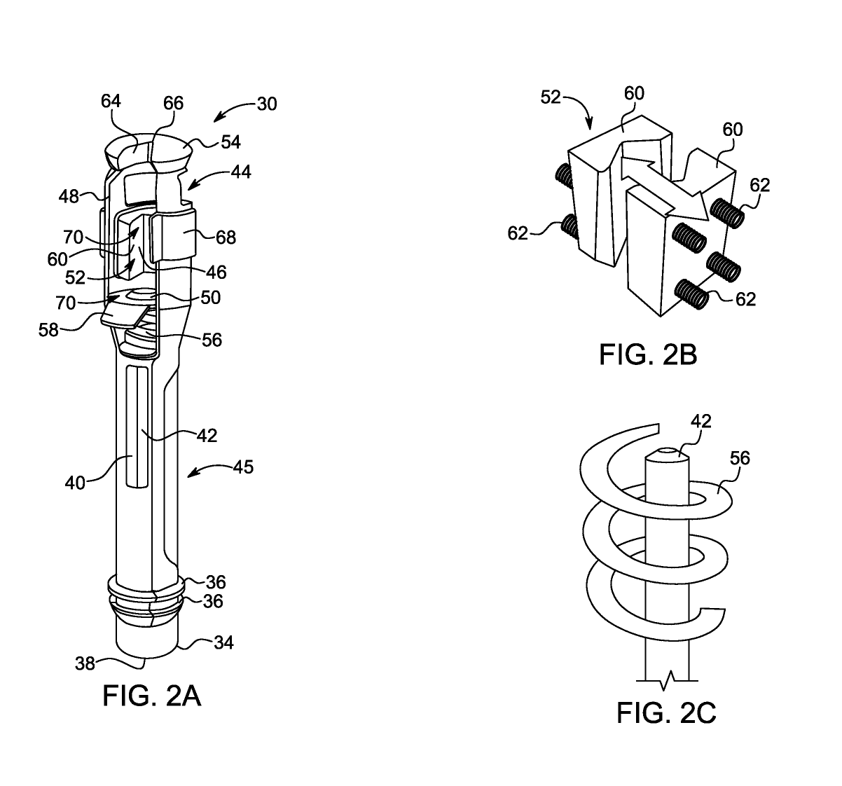

The syringe receiving portion 44 includes a cavity 46 for receiving the syringe 32. The cavity 46 is accessible through a substantially longitudinal slot 48 extending from the distal end of the adapter 30 along the syringe receiving portion 44. The cavity 46 includes an axial support or base 50, a number of radial or side supports 52, and a cap 54 covering the distal end of the adapter 30 for holding the syringe 32 in a desired position. The base 50 is a flat surface coupled to a spring 56 and moveable through the cavity 46 as a result of compression or extension of the spring 56. The base 50 includes a lip 58 extending through the slot 48. Pushing downward on the lip 58 or other portion of the base 50 compresses the spring 56 to facilitate insertion of the contrast syringe 32 into the cavity 46. When downward pressure is released, the spring 56 pushes the base 50 and syringe 32 in the distal direction relative to the adapter 30, thereby pressing the distal end 28 of the syringe 32 against the interior surface of the cap 54 to restrict axial movement of the syringe 32. The drive rod 42 extends through the spring 56 and base 50 into the cavity 46. In the cavity 46, the distal end of the drive rod 42 engages the proximal end of the plunger 18, as described above. In a preferred and non-limiting embodiment, as shown in FIG. 2C, the drive rod 42 extends through the center of the helical spring 56 to contact the plunger 18 (not shown in FIG. 2C).

With continued reference to FIGS. 1, 2A, and 3A, in a preferred and non-limiting embodiment, the side supports 52 are opposing blocks with v-shaped inward surfaces, referred to hereinafter as v-blocks 60, that define a notch configured to press against the syringe barrel 22. The v-blocks 60 are inwardly biased into the cavity 46 by side springs 62. The v-blocks 60 align the syringe 32 in an upright position, such that a longitudinal axis of the syringe 32 is aligned with a longitudinal axis of the adapter 30. The v-blocks 60 are adapted to hold syringes with different diameters. Particularly, when a narrow syringe is used, the side springs 62 push the v-blocks 60 farther into the cavity 46 to contact the syringe barrel 22. For syringes with a wider diameter, the v-blocks 60 do not extend as far into the cavity 46. The base spring 56 and the side springs 62 may be compatible for use in close proximity to a magnetic resonance imaging (MRI) machine. For example, the springs 56, 62 may be formed from a shape memory polymer or similar flexible non-metallic material, as well as MRI compatible metallic materials.

The cap 54 is a circular structure covering the open distal end of the adapter 30. The cap 54 may include a wedge-shaped slot 64 removed therefrom for receiving the nozzle 26 of the syringe 32. The cap 54 may also include a circular or curved opening 66 at the center of the cap 54 that receives and holds the nozzle 26 in an upright position. The proximal surface of the cap 54 may also include additional holding structures for supporting other portions of the distal end 28 of the syringe 32, such as the shoulder portion or end of the syringe barrel 22.

The adapter 30 further includes a latch 68 formed from a semi-annular band or ring that surrounds part of the syringe receiving portion 44 of the adapter 30. In an open position, the latch 68 does not cover the slot 48. Once the contrast syringe 32 is loaded into the cavity 46, an operator rotates or twists the latch 68 about the adapter 30 and across the slot 48 so that it covers the slot 48. Positioning the latch 68 to cover the slot 48 ensures that the operator does not prematurely remove the syringe 32 from the injector 10 before the injection is completed.

With continued reference to FIGS. 1, 2A, and 3A, the adapter 30 may include one or more sensors 70 that measure physical dimensions of the syringe 32 based on the position of the base 50 and side supports 52. The sensors 70 may be any sort of pressure or optical sensor, as is known in the art, for measuring displacement of the base 50 and side supports 52. Alternatively, pressure or loading sensors may be configured to measure compression of the springs 56, 62 to determine the syringe 32 dimensions. In a further embodiment, sensors (not shown) may be positioned in the drive rod housing 45 portion of the adapter 30 and configured to measure displacement of the drive rod 42. Information about the fluid volume contained in the syringe 32 and fluid volume expelled from the syringe 32 to the patient can be determined based on the drive rod 42 position data obtained by the sensors. Information about the physical dimensions and drive rod 42 position of the syringe 32 may be used to identify the type of syringe 32 being used for a particular procedure. Once the type of syringe 32 is identified, additional physical parameter information, including syringe fluid volume, barrel/plunger friction characteristics, pressure limitations, and maximum or minimum flow rates may be obtained. For example, the information may be downloaded to the injector 10 from a central database or computer server via a computer network. The physical parameter information about the syringe 32 may be used to determine a preferred injection force, injection velocity, and appropriate power level for the linear actuator of the injector 10.

With reference to FIGS. 3A-3C, steps for loading the contrast syringe 32 to the adapter 30 are now discussed. As shown in FIG. 3A, a proximal open end of the contrast syringe 32 is pressed against the lip 58 of the base 50. The operator presses down on the syringe 32 to compress the spring 56 connected to the base 50, thereby moving the base 50 in the proximal direction. When the base 50 is moved a sufficient amount, the operator slides the syringe 32 into the cavity 46 and presses against the notch of the v-blocks 60 extending therein until the syringe 32 is in a substantially upright position, as shown in FIG. 3B. After the syringe 32 is in the desired position, the operator rotates the latch 68 across the slot 48 to prevent the syringe 32 from being removed from the adapter 30, as shown in FIG. 3C. Once the syringe 32 is in place, the sensors 70 (shown in FIG. 2A) may be used to determine the size and capacity of the syringe. Once this information is known, the drive rod 42 may be advanced in the distal direction toward the syringe plunger 18 with the injector linear actuator. The distal end of the drive rod 42 contacts and engages the proximal end of the plunger 18 to form a suitable connection therewith. Continued distal movement of the linear actuator advances the drive rod 42 and plunger 18 attached thereto through the syringe barrel 22 to eject fluid from the syringe 32 through its nozzle 26.

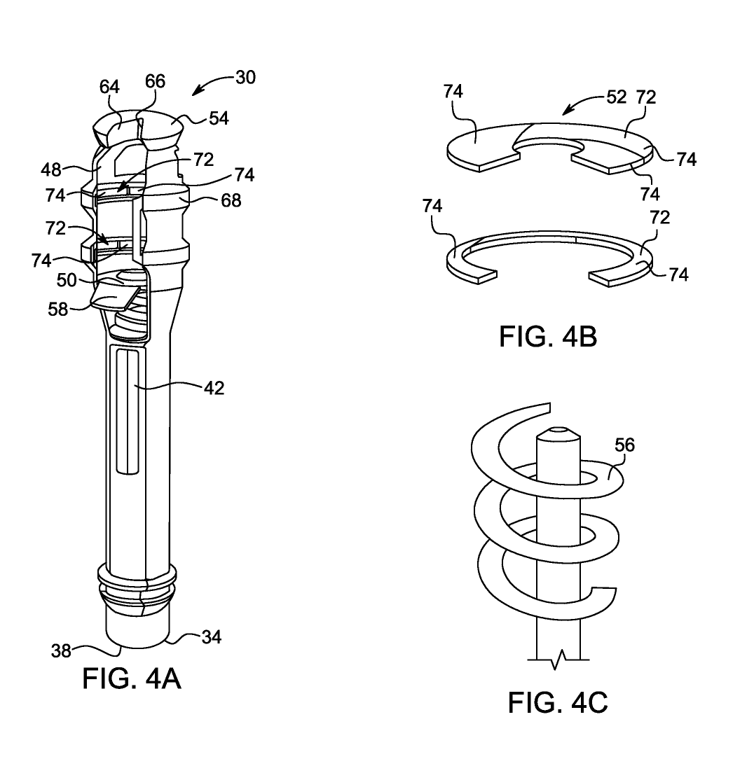

With reference to FIGS. 4A to 4C, a further embodiment of a universal adapter 30 is illustrated. The side supports 52 of the adapter 30 are an iris axial securing mechanism, including one or more semi-annular supports 72 composed of moveable segments 74. The segments 74 are configured to move radially inward or outward in a coordinated manner, similar to movement of a camera aperture, to increase or decrease the width of the semi-annular support 72. The segments 74 may be biased by one or more springs (not shown), such that the segments 74 press against the syringe 32 to restrict radial movement thereof. As shown in FIG. 4A, the semi-annular supports 72 may be positioned at different areas of the syringe holding cavity 46. For example, one semi-annular support 72 may be located near the distal end of the cavity 46 to contact and hold the nozzle 26 of the syringe 32. A second semi-annular support 72 may be located at an intermediate position of the cavity 46 and adapted to contact the wider syringe barrel 22. As in the previously described embodiment, the adapter 30 may include one or more sensors 70 configured to determine the position of the segments 74 and semi-annular supports 72.

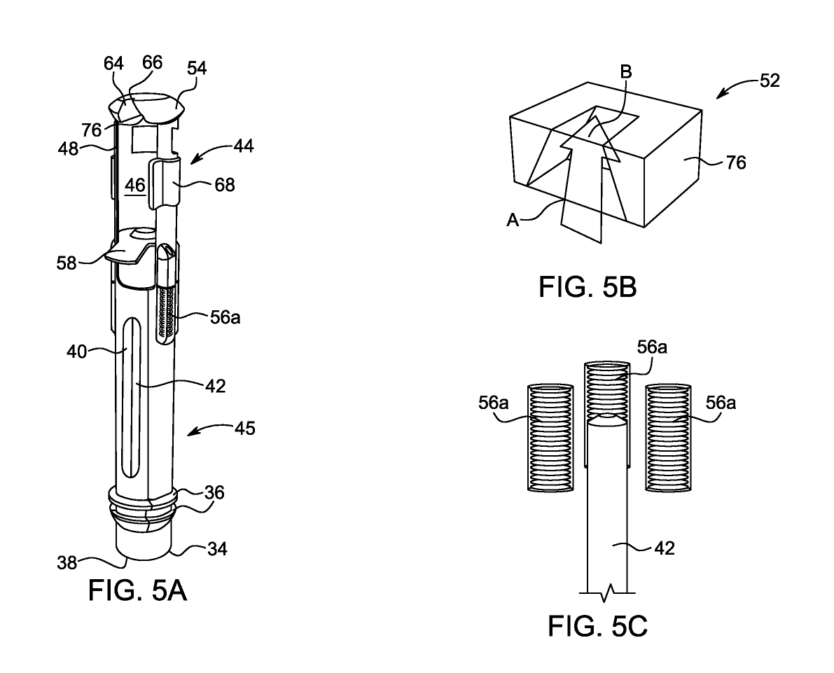

With reference to FIGS. 5A to 5C, a further embodiment of a universal adapter 30 is illustrated. The adapter 30 includes a luer lead-in support 76 attached to or integrally formed with the proximal surface of the cap 54. The luer lead-in support 76 defines a tapered cavity 78 with a wider proximal opening of width A and a narrow distal opening of width B. The tapered cavity 78 is accessible through a longitudinal slot 80 positioned to correspond to the slot 48 of the adapter 30. The tapered cavity 78 is adapted to receive the nozzle 26 of the contrast syringe 32. As a result of the tapered shape, the cavity 78 restricts both radial and axial movement of the syringe 32. The embodiment of the adapter 30 illustrated in FIGS. 5A to 5C also includes a plurality of external springs 56a coupled to the base 50. As was the case with the spring 56 of previous embodiments, the external springs 56a exert a force against the base 50 of the cavity 46. The base 50 pushes the syringe 32 in the distal direction causing the nozzle 26 and distal portion of the barrel syringe 22 to contact the luer lead-in support 76. Pressure exerted on the syringe 32 by the base 50 and the luer lead-in support 76 maintains axial and radial alignment of the syringe 32.

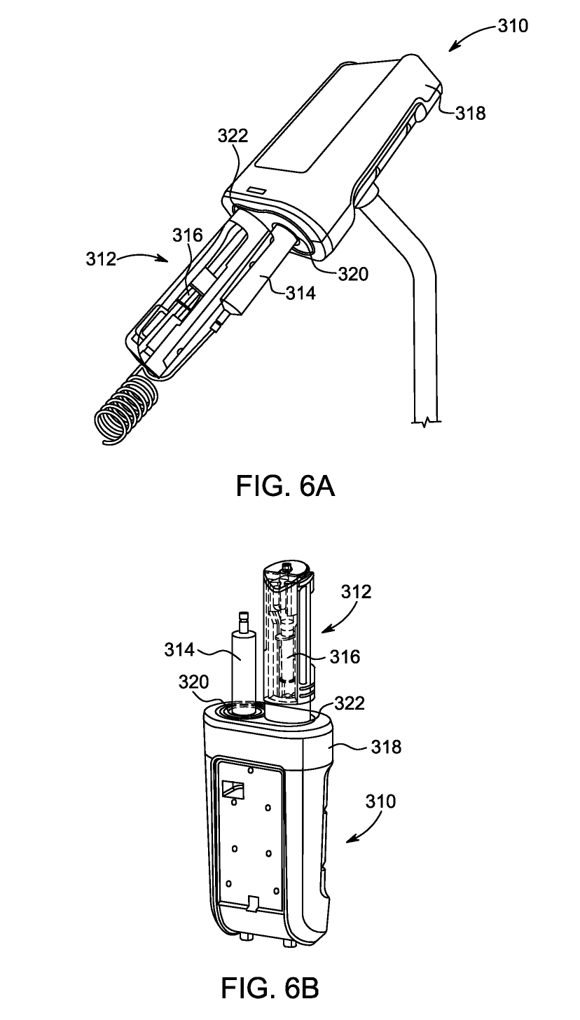

With reference to FIGS. 6A and 6B, a further embodiment of a fluid injector 310 and a universal adapter 312 is illustrated. As in previously described embodiments, the injector 310 is configured to be connected with one or more syringes, such as a saline syringe 314 and/or a contrast syringe 316, to expel fluid therefrom. The syringes 314, 316 are coupled to respective piston element of a linear actuator enclosed within a housing 318 of the injector 310 through syringe ports, such as a saline syringe port 320 and contrast syringe port 322. The syringe ports 320, 322 are located on a top side of the housing 318. In certain embodiments, the saline syringe 314 is connected directly to the saline syringe port 320. The contrast syringe 316 is connected to the contrast syringe port 322 through the universal adapter 312. The universal adapter 312 is configured to hold syringes of various geometries and dimensions.

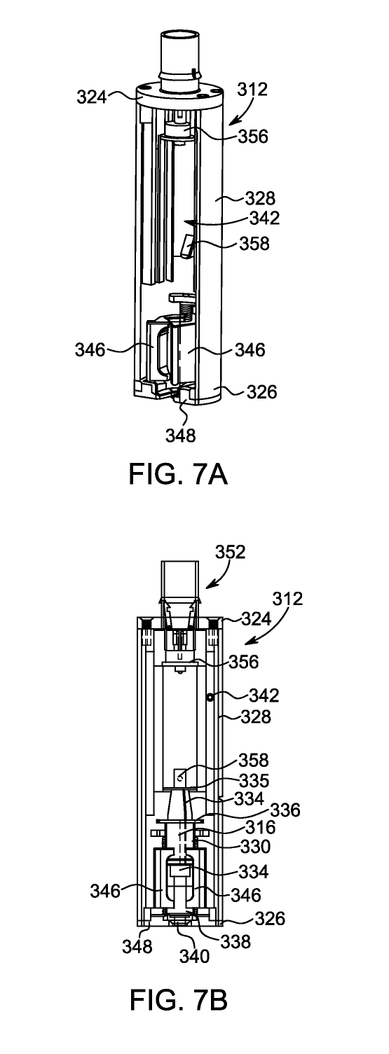

With reference to FIGS. 7A and 7B, the universal adapter 312 is a substantially tubular structure or housing having a proximal end 324 configured for insertion in the contrast syringe port 322 (shown in FIGS. 6A and 6B), a distal end 326 configured for connection with a fluid delivery assembly such as IV tubing or a needle assembly (not shown), and a cylindrical sidewall 328 extending therebetween. As shown in FIG. 7B, the adapter 312 is configured to be loaded with a fully assembled contrast syringe 316 including a syringe barrel 330, plunger 332, and plunger rod 334 extending from a proximal end 336 the barrel 330. The syringe barrel 330 also includes a distal end 338 having a nozzle 340 extending therefrom.

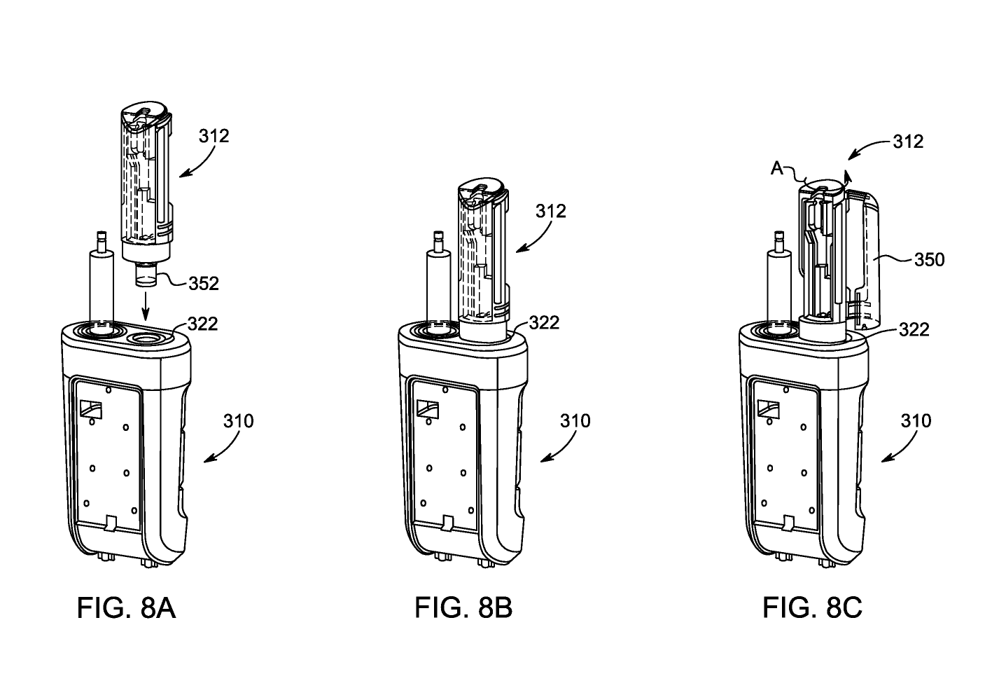

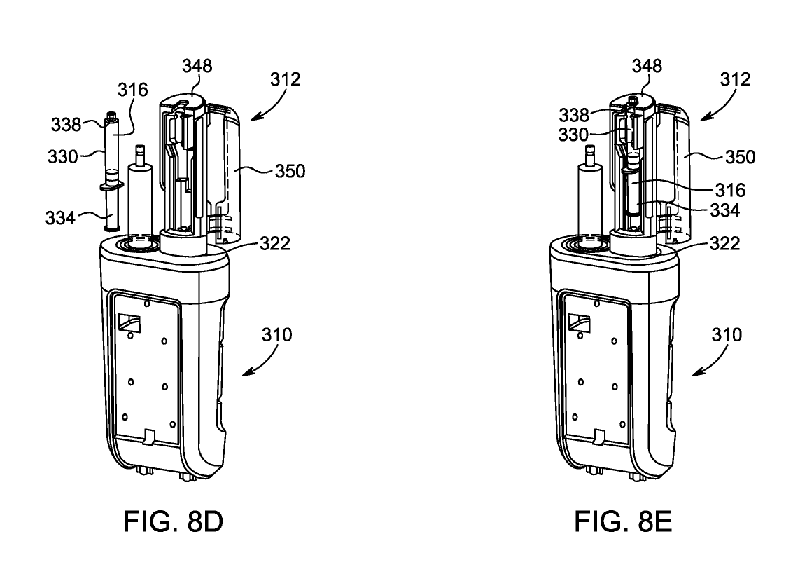

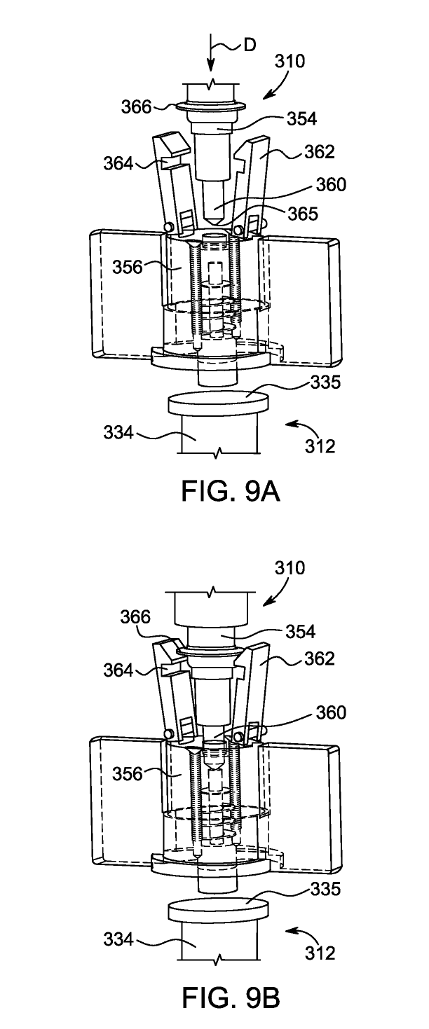

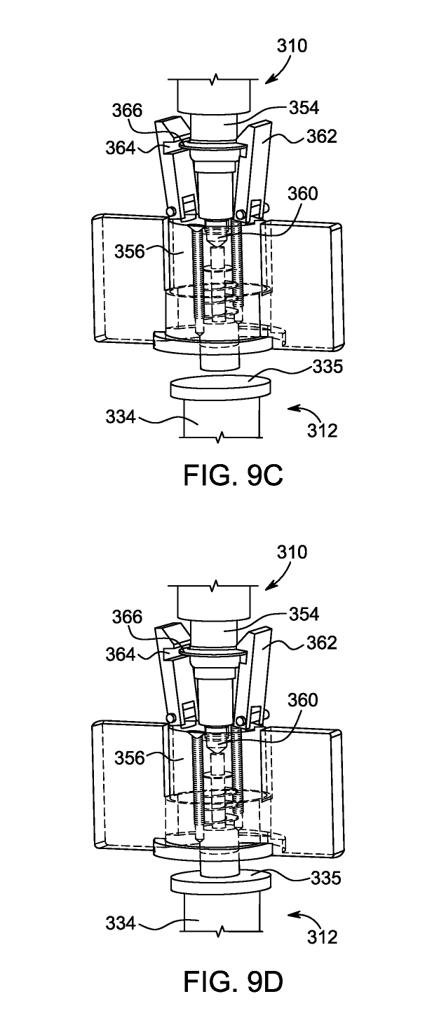

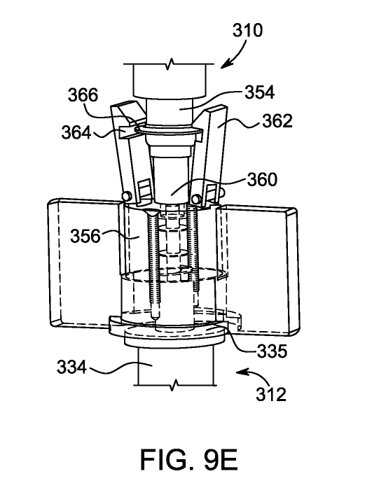

With continued reference to FIGS. 7A and 7B, the adapter 312 includes a cavity 342 for receiving the contrast syringe 316. The cavity 342 is accessible through a substantially longitudinal opening 344 extending the length of the adapter 312. The cavity 342 includes an adapter drive member assembly 356 configured to contact a syringe flange 335 located at the proximal end of the syringe plunger rod 334. As will be discussed hereinafter, the cavity 342 includes at least one side support 346 and a forward biasing mechanism 358 for holding the syringe 316 against a load plate 348 covering the distal end 326 of the adapter 312, thereby maintaining the syringe 316 in a desired position (e.g., for restricting radial and axial movement of the syringe 316 during an injection). The adaptor cavity 342 can be closed by swinging a door 350 (shown in FIGS. 8A-8E) across the longitudinal opening 344. The door 350 may be formed from a transparent or translucent material so that a user or operator can see whether the syringe 316 is loaded in the adapter 312. Additionally, to facilitate connection with the contrast syringe port 322, in certain embodiments, the adapter 312 includes an injector interface structure 352. As will be described hereinafter, during an injection procedure, a piston 354 (shown in FIGS. 9A-9E) driven by the linear actuator of the injector 310 is configured to contact and advance the adapter drive member assembly 356 toward the syringe plunger rod 334.

As shown in FIGS. 8A-8E, the operator begins the injection process by inserting the universal adapter 312 into the contrast syringe port 322 of the injector 310. In certain embodiments, the operator may be required to press the adapter 312 into the port 322, causing an interface structure 352 to engage a corresponding mounting structures (not shown) in the syringe port 322. As shown in FIG. 8C, the operator then opens the door 350, such as by swinging the door 350 in direction A. As shown in FIGS. 8D and 8E, the operator then inserts the contrast syringe 316 into the adapter 312, such that the distal end 338 of the syringe barrel 330 is pressed against the load plate 348 of the adapter 312 and the plunger rod 334 of the syringe 316 is positioned adjacent to the drive member assembly 356 (shown in FIGS. 7A and 7B). The operator then closes the door 350 and may activate the injector 310 to begin the injection.

With reference to FIGS. 9A-9E, the interface structure 352 (shown in FIG. 8A) between the drive member assembly 356 and linear actuator or piston 354 of the injector 310 will now be discussed in detail. The interface structure is intended to provide structural support for the adapter 312 to counteract forces imparted on the adapter 312 by the injector piston 354. The interface structure also maintains axial alignment between the adapter 312 and piston 354. In certain embodiments, the interface structure may be orientation independent relative to the injector 310. Thus, the interface structure may be configured to connect the adapter 312 to the injector 310 without requiring the operator to orient the adapter 312 in the syringe port 322 in any particular manner.

The interface structure may include latching members 362 extending in a proximal direction from the proximal end of the drive member assembly 356 configured to engage a portion of the piston 354. The latching members 362 may be flexible or hinged legs intended to interact with the piston 354 in a manner than causes a positive engagement therewith. The positive engagement between the latching members 362 and piston 354 causes the drive member assembly 356 to move in conjunction with motion of the piston 354 in both the advance (e.g., distal D) and retract (e.g., proximal P) directions. In certain embodiments, the latching members 362 may include a groove 364 configured to receive a corresponding shoulder or rib 366 of the piston 354. The latching members 364 are biased to deflect out of the way as the piston 354 is advanced towards the latching members 362, in the distal direction D, and then to return to an initial position to grasp the rib 366 of the piston 354. Continued distal movement of the piston 354 causes the adapter drive member assembly 356 to contact the syringe flange 335 of the syringe plunger rod 334.

More specifically, in use, the linear actuator or piston 354 advances in the distal direction as shown in FIG. 9A. Continued distal movement of the piston 354 causes the latching members 362 to deflect radially outward so that the rib 366 of the piston 354 advances past the proximal end of the latching members 362. Once the rib 366 passes the proximal ends of the latching members 362, the latching members 362 return to their initial position, such that the rib 366 is received within the groove 364 of the latching members 362. In this position, as shown in FIG. 9C, the linear actuator piston 354 is docked to the drive member assembly 356 meaning that a distal tip 360 of the piston 354 is in contact with the drive member assembly 356. Continued distal movement of the piston 354 advances the drive member assembly 356 which, in turn, contacts and advances the syringe plunger rod 334.

In certain embodiments, the distal tip 360 or proximal end of the drive member assembly 356 may include a sensor 365, such as a pressure or contact sensor, which identifies when contact between the piston 354 and drive member assembly 356 is established. The sensor 365 may be a spring loaded pin sensor that retracts when the piston 354 contacts the proximal surface of the drive member assembly 356.

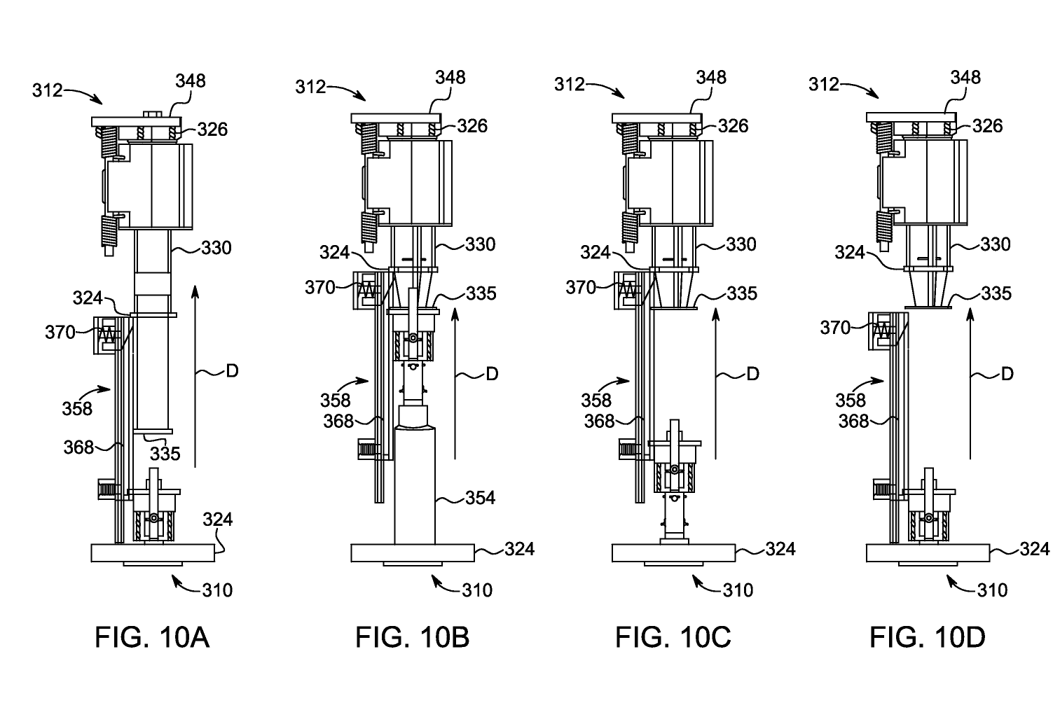

With reference to FIGS. 10A-10D, the axial or forward biasing mechanism 358 for pressing the distal end 338 of the syringe barrel 330 against the load plate 348, in distal direction D, will now be discussed in detail. The forward biasing mechanism 358 includes a biasing member 368 extending in a longitudinal direction from the proximal end 324 of the adapter 312 to the distal end 326. The biasing member 368 is engaged to the adapter drive member assembly 356 through a biased detent 369, such as a spring plunger ball. A tab 370 is connected to the distal end of the biasing member 368. The tab 370 is configured to press against the proximal end 324 of the syringe barrel 330 to provide the forward biasing force that presses the syringe barrel 330 against the load plate 348. In certain embodiments, the tab 370 is spring loaded to accommodate different flange and plunger rod dimensions.

As shown in FIG. 10A, in its initial position, the detent 369 connects to the adapter drive member assembly 356 and the tab 370 contacts and presses against the proximal end 336 of the syringe barrel 330. As the piston 354 (shown in FIG. 10B) of the injector 310 advances the drive member assembly 356 through the adapter 312, the detent 369 deflects away from the drive member assembly 356 releasing the engagement therebetween. Once the detent 369 releases from the drive member assembly 356, the biasing member 368 remains stationary maintaining forward biasing pressure against the tab 370, as the drive member assembly 356 advances further through the adapter 312 to eject fluid from the syringe 316.

As shown in FIG. 10B, when the injection is completed, the distal end of the drive member assembly 356 is adjacent to the tab 370 and the proximal end 336 of the syringe barrel 330. As shown in FIG. 10C, after the injection is completed, the piston 354 retracts in a proximal direction causing the drive member assembly 356 to move toward the proximal end of the adapter 312. As the drive member assembly 356 retracts, the tab 370 is deflected out of the way by the syringe flange 335. Accordingly, the biasing member 368 and tab 370 can be used with syringes 316 having a syringe flange 335 that is wider than the syringe barrel 330.

With reference to FIG. 10D, further retraction of the drive member assembly 356 causes the assembly 356 to approach the detent 369 located at the proximal end of the biasing member 368. The drive member assembly 356 contacts and engages the detent 369. As shown in FIG. 10D, once the engagement between the drive member assembly 356 and detent 369 is reestablished, the biasing member 368 moves in the proximal direction in conjunction with the drive member assembly 356. Thus, the biasing member 368 ultimately returns to its initial position, in which a distal end of the biasing member 368 is positioned against the proximal end 324 of the adapter 312. In this position, the empty syringe 316 can be easily removed from the adapter 312.

By biasing the syringe barrel 330 against the front load plate 348 of the adapter 312, the forward biasing mechanism 358 is useful for ensuring that the syringe zero volume position (e.g., the position of the injector piston 354 when all fluid has been ejected from the syringe barrel 330) is accurately established. Accurately establishing the syringe zero volume position means that fluid volume measurements can be determined based on absolute position of the injector piston 354. If the syringe zero volume position could not be accurately established, then such volume measurements could not be determined based on the position of the injector piston 354 and would need to be directly measured using some other volume sensor positioned elsewhere in the adapter 312 or syringe 316.

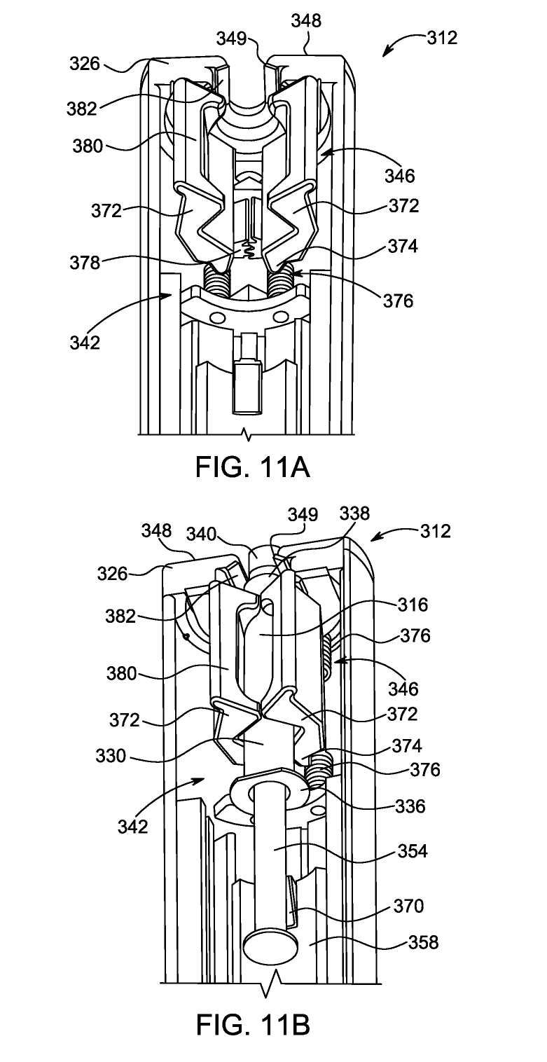

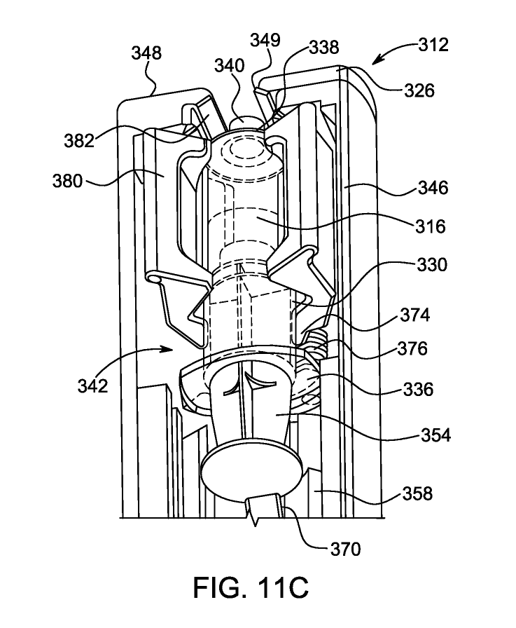

With reference to FIGS. 11A-11C, the structure of the radial or side supports 346 of the adapter 312 will now be discussed in detail. As in previously described embodiments, the side supports 346 may be v-shaped blocks 372 that define a notch for receiving the syringe. The v-shaped blocks 372 are configured to restrict radial movement of the syringe barrel 330. The “v” shape allows the blocks 372 to hold syringe barrels having different diameters. It is noted that while the adapter 312 illustrated in FIGS. 11A-11C is shown with two v-shaped blocks 372, an adapter 312 including only a single v-shaped block can also be constructed. In that case, the single block 372 or side support 346 biases the syringe toward a receiving surface, such as a protrusion or padded region of the interior of the housing. Pressure exerted on the syringe by the single block 372 and receiving surface maintains the syringe in the desired alignment.

In some embodiments, the v-shaped blocks 372 are connected to the adapter 312 at a hinge 374 and are maintained in position by one or more biasing members, such as torsion springs 376. The torsion springs 376 allow the v-shaped blocks 372 to be deflected away from the opening or slot of the adapter 312, so that the syringe barrel 330 can be inserted into the adapter 312. The torsion springs 376 return the v-shaped blocks 372 to their initial position, contacting a portion of the syringe barrel 330, once the syringe barrel 330 is inserted in the adapter 312. In certain embodiments, the v-shaped blocks 372 are coupled to a gear mechanism 378 (shown in FIG. 11A) to ensure that the blocks 372 open and close in conjunction with one another. As shown in FIGS. 11A-11C, the v-shaped blocks 372 may also include an outwardly flared or outwardly bending portion 380 that aligns with the longitudinal opening 344 (shown in FIGS. 1A and 1B) of the adapter 312 and assists a user to slide the syringe barrel 330 into the cavity 342 of the adapter 312.

As previously described, the load plate 348 is positioned at the distal end 326 of the adapter 312. The load plate 348 is a generally flat surface that includes an aperture 349 configured to receive the nozzle 340 of the syringe 316. The load plate 348 may include a riser 382 or stepped portion. The riser 382 creates a space or gap between the distal end 338 of the syringe barrel 330 and the load plate 348 so that, even when a shorter syringe is being used, the syringe 316 still extends beyond the proximal end of the v-shaped blocks 372. As shown in FIGS. 11B and 11C, when the proximal end 336 of the syringe barrel 330 extends past the proximal end of the v-shaped blocks 372, the drive member assembly 356 can advance all the way to the syringe zero volume position. If this were not the case, then an amount of fluid would be left in the syringe 316 after the injection is completed.

With continued reference to FIGS. 11A-11C, in use, the operator presses the syringe barrel 330 against the outwardly bending portions 380 of the v-shaped blocks 372. Pushing the syringe barrel 330 into the cavity 342 deflects the v-shaped blocks 372 away from the longitudinal axis of the cavity 342, allowing the user to push the syringe 316 past the outwardly bending portions 380 and into the cavity 342. Once the syringe barrel 330 is pushed past the outwardly bending portions 380, the torsion springs 376 drive the v-shaped blocks 372 to return to their initial position. In this initial position, the blocks 372 surround at least a portion of the syringe barrel 330 to prevent the syringe barrel 330 from moving in the radial direction. In this position, the distal end 338 of the syringe barrel 330 is pressed against the front load plate 348 by the tab 370 of the forward biasing mechanism 358. A syringe 316 which has been inserted into the cavity 342 and is in a ready for use position is illustrated in FIG. 11B. From this position, the piston 354 of the injector (not shown in FIGS. 11A-11B) may advance the syringe plunger rod 334, to eject fluid from the syringe 316. A syringe in a completed injection position is illustrated in FIG. 11C.

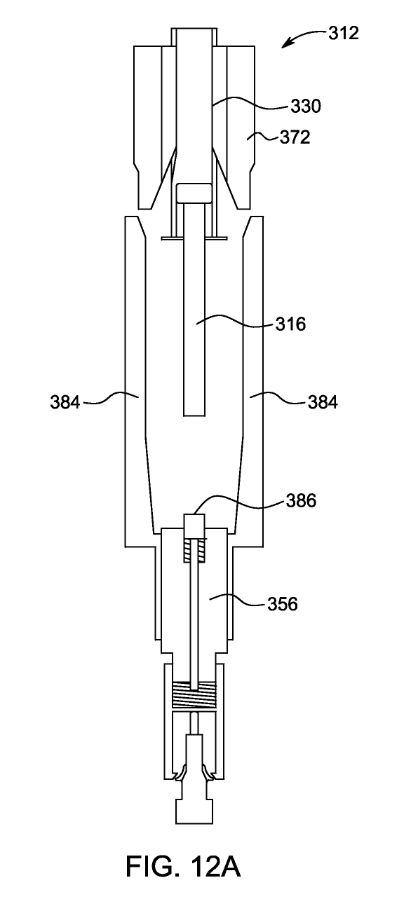

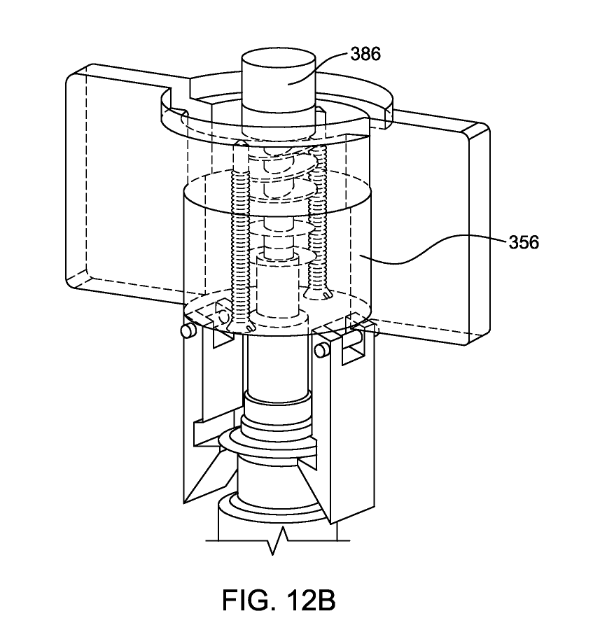

As in previously described embodiments, the adapter 312 may include sensors for measuring the physical dimensions of the syringe 316. For example, with reference to FIGS. 12A and 12B, the adapter 312 may include an outer diameter detector 384 configured to measure displacement of the v-shaped blocks 372. As shown in FIG. 12A, the outer diameter detector 384 is coupled to the adapter plunger rod assembly 356. As the adapter drive member assembly 356 advances through the adapter 312, the outer diameter detector 384 contacts an outer surface, such as a chamfered surface, of the v-shaped blocks 372 to measure the position of the blocks 372. The wider the diameter of the syringe barrel 330 inserted in the v-shaped blocks 372, the farther apart the corresponding members of the outer diameter detector 384 are pushed from one another. A sensor (not shown), such as a contact sensor or optical sensor, may be positioned adjacent to the outer diameter detector 384 for measuring the displacement of the detector 384. It is noted that by measuring the position of the v-shaped blocks 372 rather than the diameter of the proximal end 336 of the syringe barrel 330 that extends beyond the blocks 372, the detector 384 is able to accurately measure the syringe barrel diameter even if the proximal end 336 of the barrel or syringe flange 335 is wider than the barrel 330 itself.

A plunger detector 386, such as a contact or pressure sensor, may also be positioned on the distal end of the adapter drive member assembly 356. In certain embodiments, the plunger detector 386 is configured to measure the position at which the drive member assembly 356 contacts the plunger rod 334 of the syringe 316. The positioning information could be used to determine the length of the syringe barrel 330. Once the length and diameter of the syringe barrel 330 are known, the syringe barrel 330 volume can be estimated. The plunger detector 386 may also be used to measure the position of the adapter drive member assembly 356 within the adapter cavity 342. The position of the drive member assembly 356 may be used to determine the volume of fluid expelled from the syringe 316 and when the drive member assembly 356 has emptied all contents of the syringe 316, so that the syringe is in the zero volume position. In certain embodiments, the adapter 312 may only include a plunger detector 386, without the outer diameter detector 384.

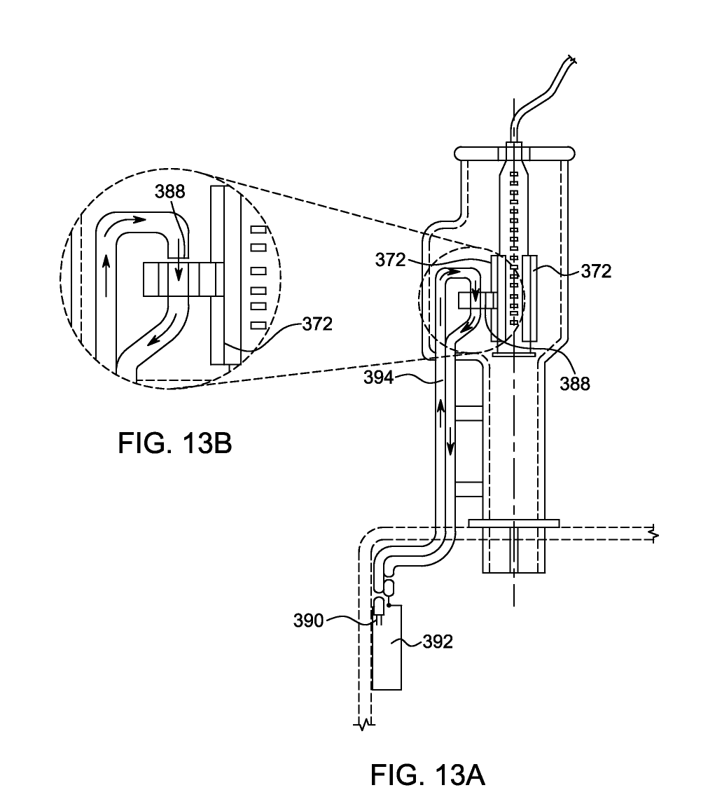

In another embodiment, with reference to FIGS. 13A and 13B, the adapter 312 may include at least one optical sensor 388 for directly measuring displacement of the v-shaped blocks 372. The optical sensor 388 may include a photo emitter 390 for directing emitted radiation toward an outward facing surface of the v-shaped block 372. The optical sensor 388 also includes a photo detector 392 for measuring the distance traveled by the emitted radiation to determine the position of the v-shaped blocks 372. In certain embodiments, fiber optical cables 394 may be used for transporting the radiation emitted by the photo emitter 390 to the v-shaped block 372 and from the block 372 to the photo detector 392. For example the fiber optic cables 394 may be in the form of one or more optical lightpipes. The lightpipes may be injection molded into the housing of the adapter 312.

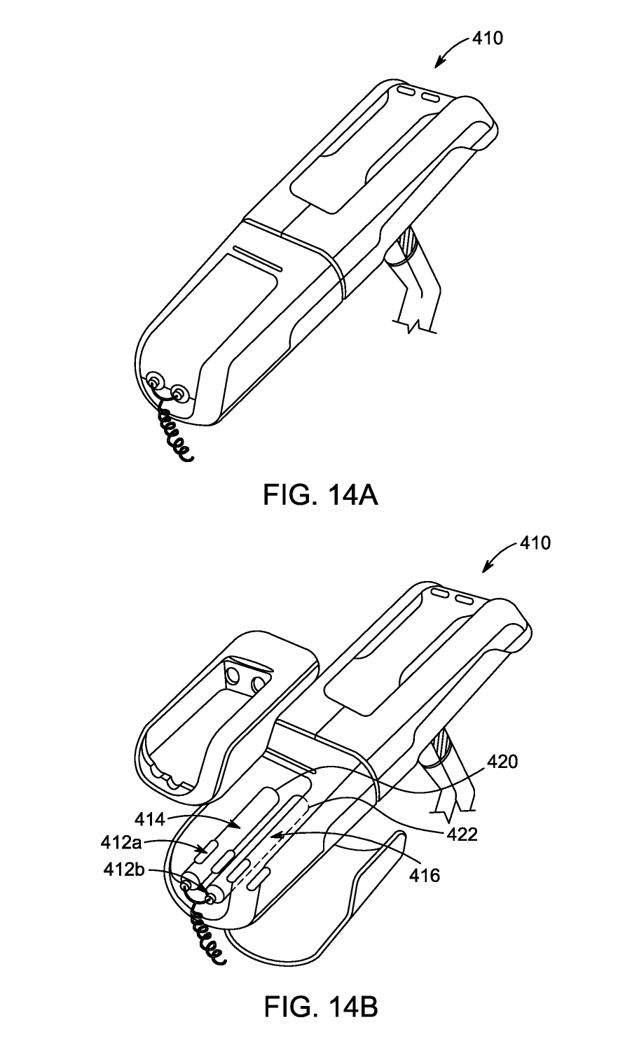

With reference to FIGS. 14A and 14B, an embodiment of a fluid injector 410 having two adapters, such as a saline adapter 412a and a contrast adapter 412b, is illustrated. The injector 410 has two syringe ports 420, 422 configured to receive two syringes, such as a contrast syringe 414 and a saline syringe 416. As in previously described embodiments, the adapters 412a, 412b are used so that different sizes and types of syringes can be attached to the injector. Using a dual adapter injector 410 allows a user to use saline syringes 414 and the contrast syringes 416 of different shapes and sizes. The adapters 412a, 412b may include sensors for determining physical dimensions and other physical parameters of the syringes 414, 416 to modify or optimize injector 410 settings for the particular types of syringes inserted in the adapters.

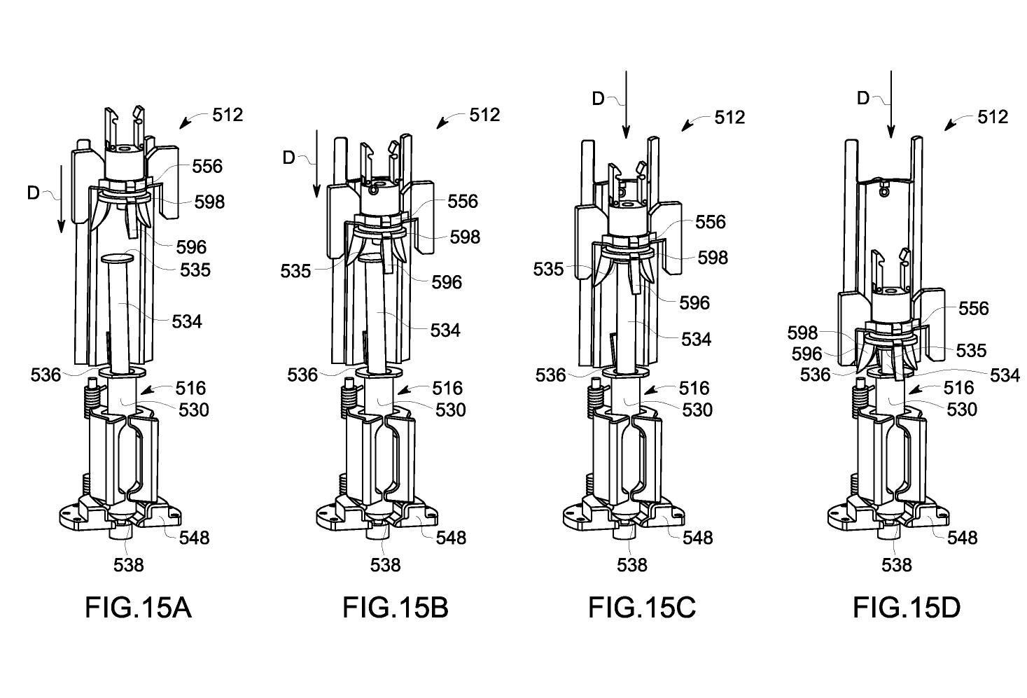

With reference to FIGS. 15A-15D, a further embodiment of an adapter 512 is illustrated. The adapter 512 includes a drive member assembly 556 for contacting and advancing a plunger rod 534 of a syringe 516, such as a prefilled syringe. The syringe plunger rod 534 extends from the proximal end 536 of the syringe barrel 530. As in the previously described embodiments of the adapter 512, the syringe 516 is received in a distal portion of the adapter 512, such that the distal end 538 of the syringe 516 is pressed against a load plate 548 of the adapter 512. The adapter drive member assembly 556 advances to contact a syringe flange 535 located at a proximal end of the syringe plunger rod 534. Continued movement of the drive member assembly 556 in the distal direction D advances a plunger or stopper through the syringe barrel 330 to expel fluid therefrom.

With continued reference to FIGS. 15A-15D, the drive member assembly 556 includes one or more flexible or pivoting fingers 596 configured to contact and engage the flange 535 on the proximal end of the syringe plunger rod 534. The fingers 596 are capable of pivoting or deflecting radially outward to accept syringe flanges 535 of the syringe plunger rod 534 with different shapes and diameters. The fingers 596 are biased by an elastic band 598, such that the fingers 596 are capable of pushing the syringe flange 535 in order to orient the plunger rod 534 concentrically with the syringe barrel 530.

In use, as shown in FIG. 15A, the syringe plunger rod 534 may, initially, be misaligned with the syringe barrel 530. As shown in FIG. 15B, the adapter drive member assembly 556 advances toward the syringe 516, causing at least one of the fingers 596 of the adapter drive member assembly 556 to contact the syringe flange 535. The elastic band 598 biases the fingers 596 to push against the syringe flange 535 to correctly orient the syringe flange 535 relative to the adapter drive member assembly 556. Continued advancement of the adapter drive member assembly 556 causes the fingers 596 to spread or deform radially outward so that the syringe flange 535 is flush against the distal end of the adapter drive member assembly 556. In certain embodiments, the adapter 512 is configured to allow the fingers 596 to deflect outward wider than the diameter of the largest syringe barrel 530 and syringe flange 535 that can be accepted by the adapter 512. Therefore, the fingers 596 have sufficient clearance to deflect around and grasp flanges 535 having unique and larger geometries without contacting or being restricted by the inner sidewall of the adapter 512.

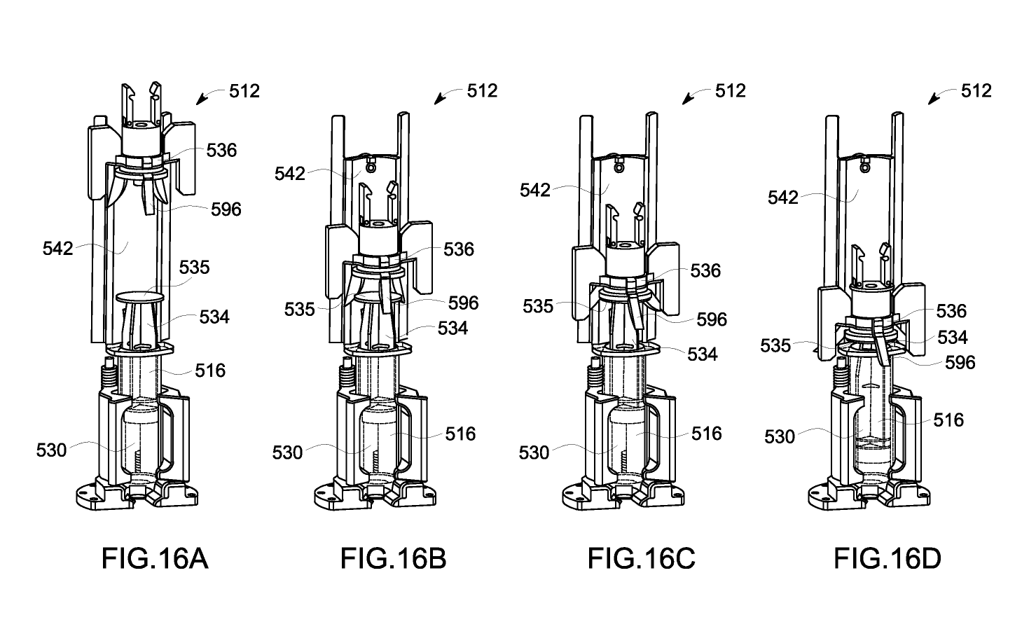

With reference to FIGS. 16A-16D, the adapter drive member assembly 556 may also be used to retract the biasing member 368 (shown in FIGS. 10A-10D) in a proximal direction through the cavity 542 of the adapter 512. As shown in FIG. 16A, the adapter drive member assembly 556 moves toward the flange 535 of the syringe 516, which is in the zero volume position. In this zero volume position, the syringe flange 535 does not require alignment, since any misalignment of the retracted syringe plunger rod 534 would clearly be minimal. As shown in FIGS. 16B and 16C, as the adapter drive member assembly 556 moves toward the syringe flange 535, the fingers 596 spread outward to allow the adapter drive member assembly 556 to contact the syringe flange 535.

Having described the structure and method of use of the fluid injector and universal adapter, a system for identifying the syringe inserted in the adapter is now discussed in detail. The system identifies the type of syringe being used for the injection and, optionally, the fluid contained therein. As will be described hereinafter, the syringe identification system may be a fully automatic system that does not require any additional activity by an operator other than connecting the syringe to the injector, a semi-automatic process that requires the operator to scan or test the syringe, or a manual system that requires the operator to identify the syringe and manually enter identification information to the system.

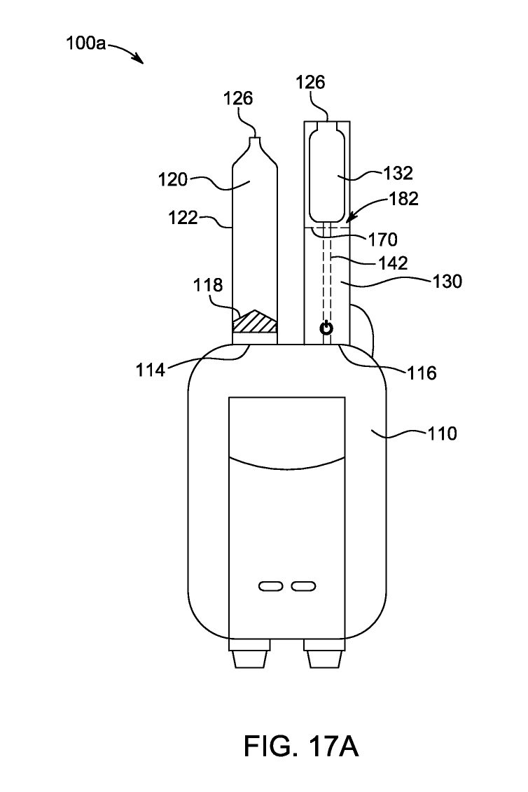

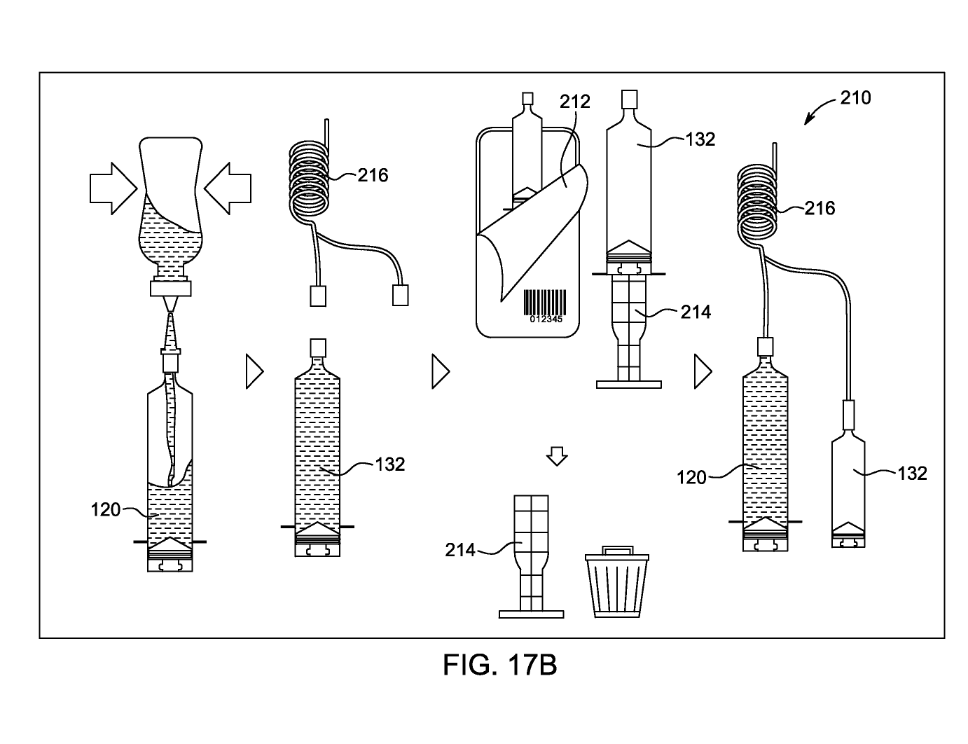

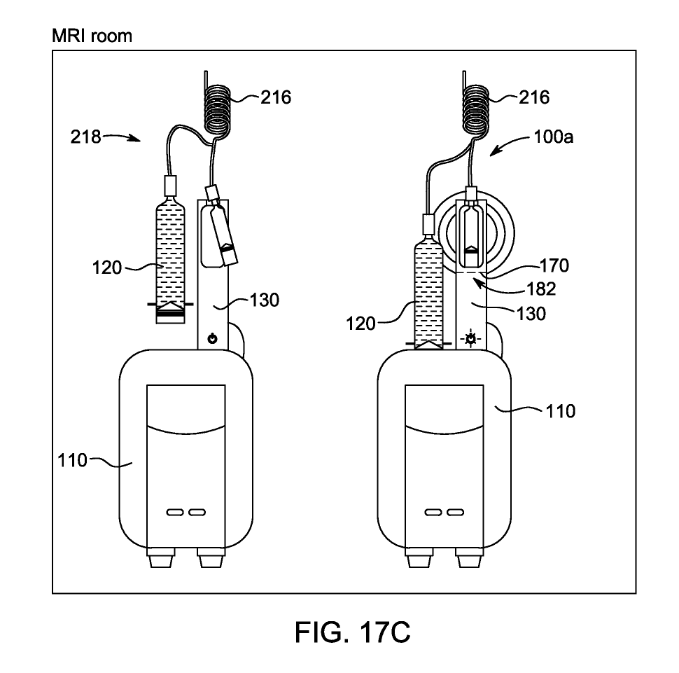

With reference to FIGS. 17A-17C, an automated system 100a for identifying the syringe 132 in a universal adapter 130 is illustrated. The automated system 100a includes a prefilled contrast syringe 132, a saline syringe 120, administration tubing 133, and a universal adapter 130 of a powered injector 110. The injector 110 includes a first port 114 for receiving the saline syringe 120 and the universal adapter 130 for receiving the contrast syringe 132. In the embodiment of FIGS. 17A-17C, the universal adapter 130 is a semi-permanent adapter that remains attached to the injector 110 before and after the injection. The adapter 130 and/or the injector 110 includes a built-in sensor array 182 formed from a plurality of sensors 170 for identifying physical characteristics of the contrast syringe 132. For example, as described above, the sensor array 182 may measure the physical dimensions of the syringe. Optionally, the sensor array 182 may also read labels, bar codes, or similar identification tags on the syringe 132 to obtain additional information about the syringe 132.

As shown in FIG. 17B, the saline syringe 120 and pre-filled contrast syringe 132 are obtained and prepared for injection in a drug preparation room, referred to hereinafter as the prep room 210. In the prep room 210, saline is transferred from a container into the saline syringe 120. The prefilled contrast syringe 132 is removed from product packaging 212 and, if necessary, a syringe plunger rod 214 is affixed thereto. The syringes 120, 132 are then connected to administration tubing 216. The syringes 120, 132 and the connected administration tubing 216 are then transported from the prep room 210 to an MRI room 218 for administration to the patient. The MRI room 218 is illustrated in FIG. 17C. In the MRI room 218, the saline syringe 120 is attached to the first port 114 of the injector 110. The contrast syringe 132 is loaded into the semi-permanent universal adapter 130, which is attached to the second port 116. Once the syringes 120, 132 are loaded into the injector 110, the sensor array 182 scans the contrast syringe 132 to identify physical dimensions and other physical parameters thereof. The sensor array 182 includes one or more optical and/or pressure sensors that identify the type of syringe 132 by measuring physical dimensions of the syringe 132. As described above, once the syringe type is determined, additional parameters about the syringe may be obtained. For example, physical parameters including barrel/plunger frictional characteristics, pressure limitations, and maximum and minimum flow rates, may be downloaded to the injector 110 from an electronic database accessible through a computer network.

Additional sensors may also be used for determining geometric dimensions of the syringe 132. For example, the adapter 130 may include sensors configured to measure portions of the syringe barrel to determine linear physical dimensions, angular dimensions, or strain/flex measurements to determine syringe geometry. Ultrasonic, optical, or imaging sensors may also be used. In addition, the adapter 130 may include a “bed of nails” arrangement in which the syringe 132 geometry is pressed into a bed of deformable or movable members. The displacement of the members is measured to determine syringe geometry. Alternatively, fluid displacement measurements or injector position measurements may also be used to determine syringe geometry.

In certain embodiments of the system 100a, the adapter 130 may communicate the syringe type and other physical parameters to controls located in the prep room 210 so that the operator can manually adjust the injector 110 settings. The system 100a may also be configured to automatically adjust the injector 110 settings based on the obtained information. For example, if the syringe size, fluid volume, or fluid type is incorrect for the procedure to be performed, the system 100a may cancel the pending injection and alert the operator about the identified discrepancies. The injection force, duration, or fluid flow rate may be altered to ensure that the correct fluid volume is delivered to the patient at a clinically appropriate rate. Information about syringe type and fluid content could also be used to update patient records, a medical facility's disposable device inventory, and for other administrative purposes.

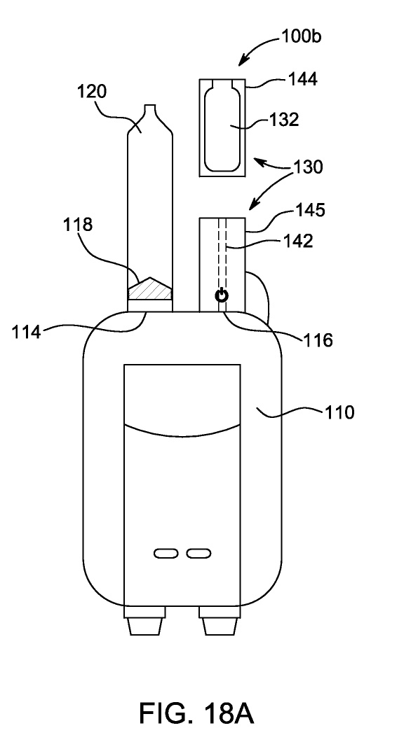

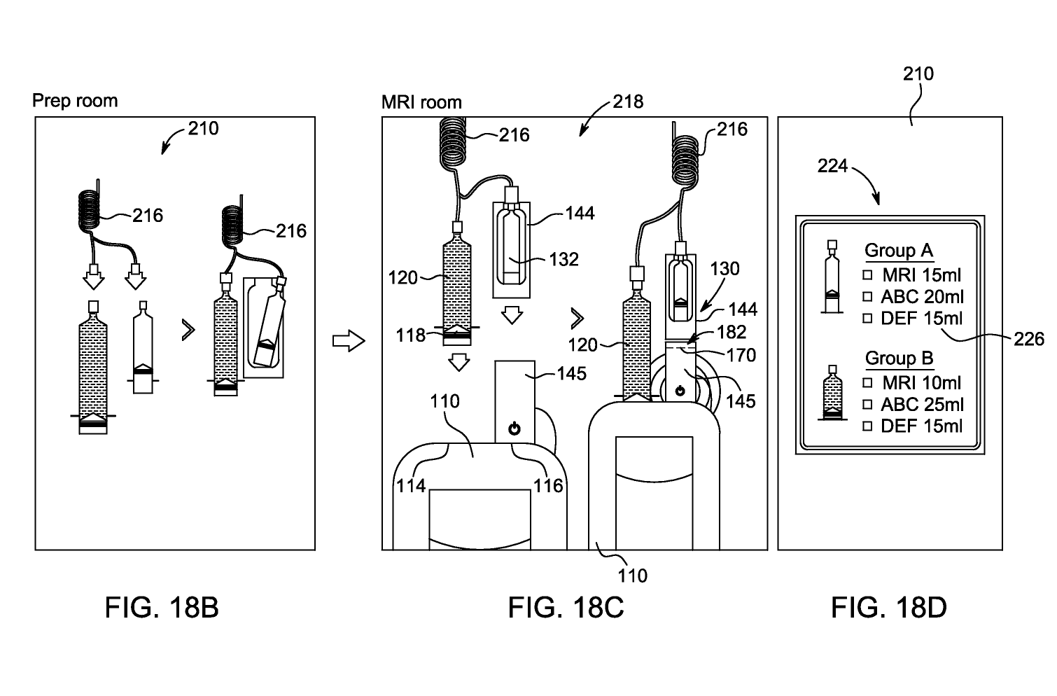

With reference to FIGS. 18A-18D, an embodiment of a semi-automatic system 100b for identifying the type of syringe 132 inserted in a universal adapter 130 is illustrated. As with the previously described automatic system, the system 100b includes a saline syringe 120, which is filled in the prep room 210, a prefilled contrast syringe 132, associated administration tubing 216, and an injector 110. As shown in FIG. 18A, the injector 110 includes a first fluid port 114 for the saline syringe 120 and a second fluid port 116 with a universal adapter 130. The adapter 130 is a two-part adapter assembly having a plunger rod housing portion 145 removeably connected to a syringe receiving portion 144. The adapter 130 includes a simple sensor array 182 made up of one or more sensors 170. As in the previous embodiment, the saline syringe 120 and the contrast syringe 132 are prepared and connected to administration tubing 216 in the prep room 210. The contrast syringe 132 is placed in the syringe receiving portion 144 of the adapter 130 in the prep room 210. The syringes 120, 132 and syringe receiving portion 144 are then transported from the prep room 210 to the MRI room 218. In the MRI room 218, the saline syringe 120 is connected to the first port 114 and the syringe receiving portion 144 of the adapter 130 is connected to the plunger rod housing portion 145, which is permanently or semi-permanently connected to the injector 110. While the simple sensor array 182 detects certain features about the contrast syringe 132, the simple sensor array 182 does not detect enough data to fully identify the syringe size and type. Instead, as shown in FIG. 18D, the system 100b identifies a list of possible syringes based on information collected by the simple sensor array 182. The operator returns to the prep room 210 and views the list of possible syringes on a visual display 226 of an electronic device 224, such as a personal computer (PC), tablet PC, or smartphone. The operator selects the correct syringe using a computer accessory, such as, a keyboard, mouse, touchscreen, or trackpad. Once the correct syringe 132 is selected, the operator may activate the injector 110 to begin the injection process.

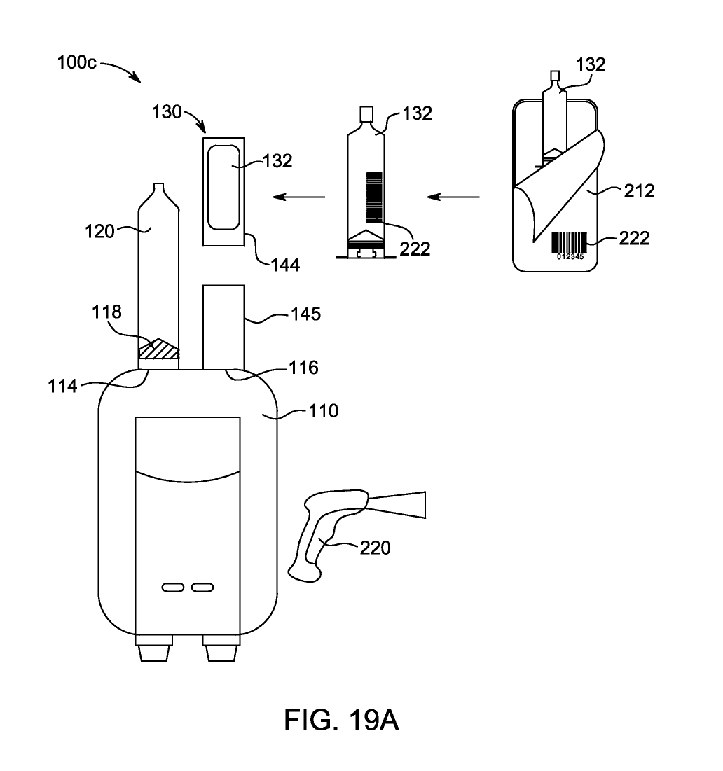

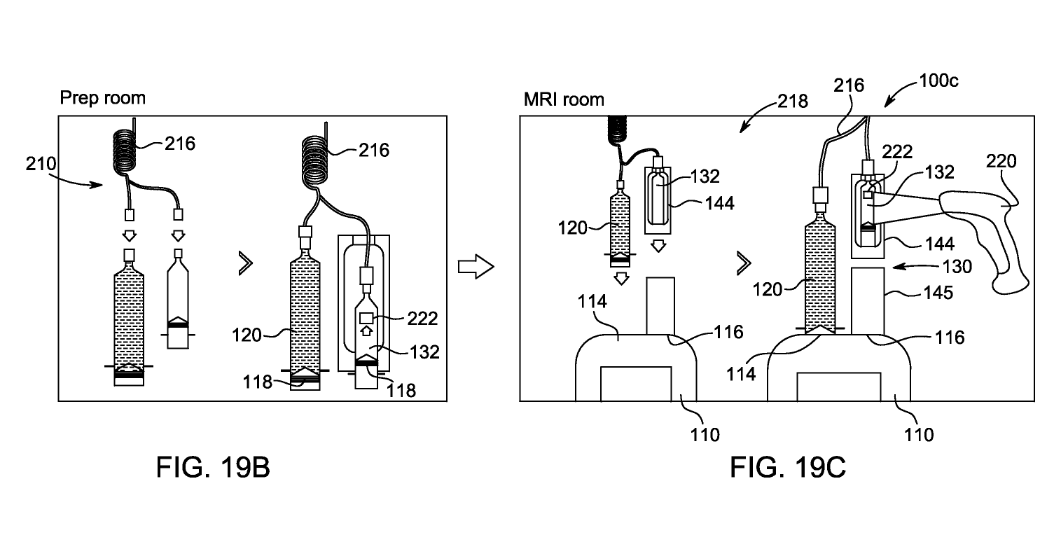

With reference to FIGS. 19A-19C, an embodiment of a semi-automatic system 100c for syringe identification is depicted. The system 100c includes the saline syringe 120, the prefilled contrast syringe 132, administration tubing 216, and the injector 110. The injector 110 includes a first port 114 for the saline syringe 120 and a two-part universal adapter 130, consisting of a syringe receiving portion 144 and plunger rod housing portion 145. The system 100c also includes a handheld scanner 220 for scanning an identification tag 222, such as a conventional one dimensional barcode, two-dimensional barcode (e.g. a QR code), or similar indicia, that is provided on or associated with the contrast syringe 132. For example, the identification tag 222 may be printed directly onto the syringe barrel 122 of the contrast syringe 132 or may be printed to a label affixed to the barrel 122. The identification tag 222 may also be attached to or printed on packaging of the syringe 132.

In use, the saline syringe 120 and the contrast syringe 132 are prepared for injection in the prep room 210. Specifically, an operator fills the saline syringe 120 with saline solution. The operator removes the contrast syringe 132 from its packaging. The operator then connects administration tubing 216 to the syringes 120, 132 and inserts the contrast syringe 132 in the syringe receiving portion 144 of the adapter 130. The syringes 120, 132, syringe receiving portion 144, and tubing 216 are then transported to the MRI room 218. In the MRI room 218, the saline syringe 120 is connected to the first port 114 and the syringe receiving portion 144 of the adapter 130 is connected to the plunger rod housing portion 145. The handheld scanner 220 is then used to read the identification tag 222. The identification tag 222 is embedded or associated with information about the syringe 132 including physical dimensions, flow characteristics, information about the fluid contained therein, and other relevant information. The system 100c may automatically check that the syringe 132 is correct for the procedure to be performed. Once the check is completed, the injection procedure is started either automatically or manually by the system 100c operator.

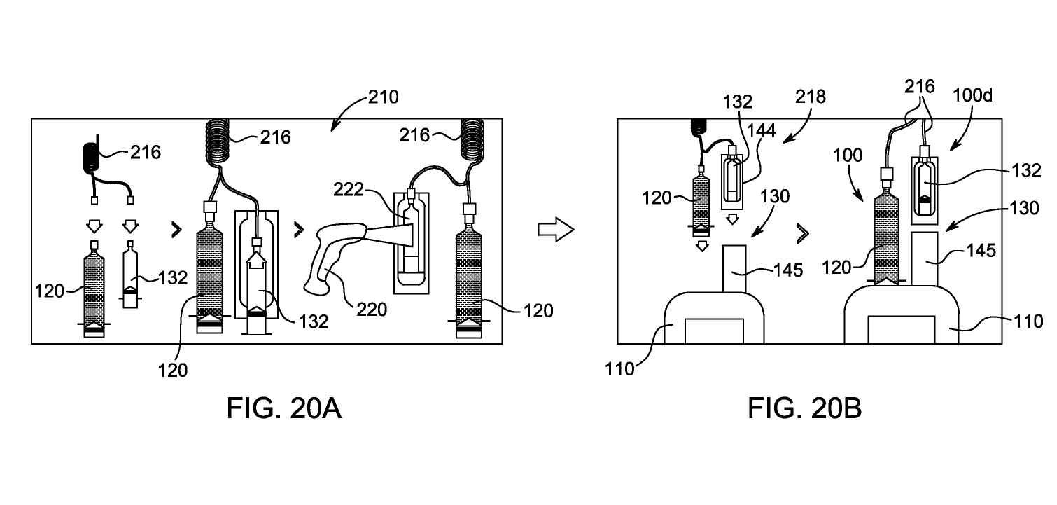

With reference to FIGS. 20A and 20B, another embodiment of the syringe identification system 100d is illustrated. The system 100d includes the same elements as the system 100c of FIGS. 19A-19C. However, in the system 100d, the operator scans the identification tag 222 in the prep room 210, rather than after the syringe 132 is connected to the injector 110. Advantageously, by scanning the identification tag 222 in the prep room 210, the syringe 132 is quickly identified and the system 100d ensures that it is correct for the procedure to be performed. If it is determined that the syringe 132 is not appropriate for a particular procedure, the operator can easily obtain a replacement syringe 132 from syringes stored in the prep room 210. Once the correct syringe is obtained and loaded to the syringe receiving portion 144 of the adapter 130, as in previous embodiments, the syringes 120, 132 and administration tubing 216 are transported from the prep room 210 to the MRI room 218 for loading to the injector 110. Once the syringes 120, 132 are loaded to the injector 110, the injection procedure is manually or automatically started.

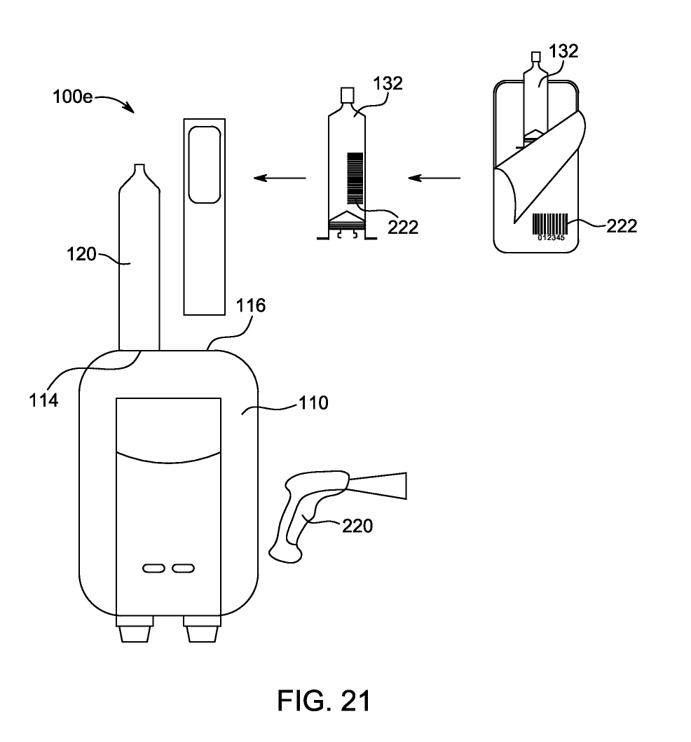

With reference to FIG. 21, a system 100e including a single-part adapter 130, in which the syringe receiving portion 144 and plunger rod housing portion 145 are integrally formed, and handheld scanner 220 is illustrated. As in the previously described embodiments, an operator inserts a contrast syringe 132 into the adapter 130. The operator then scans an identification tag 222 included on the syringe 132 or syringe packaging 212 using the scanner 220. The operator may scan the identification tag 222 either in the prep room (not shown in FIG. 21) or in the MRI room (not shown in FIG. 21). The adapter 130 is transported from the prep room to the MRI room. In the MRI room, the entire adapter 130, including the syringe receiving portion 144 and plunger rod housing portion 145, are connected to the second port 116 of the injector 110.

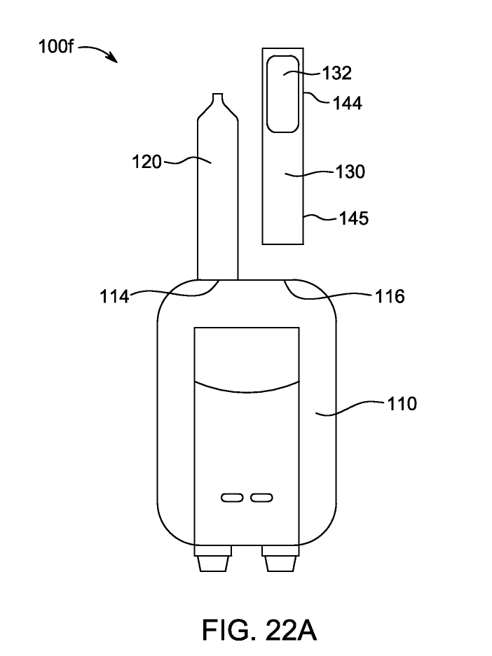

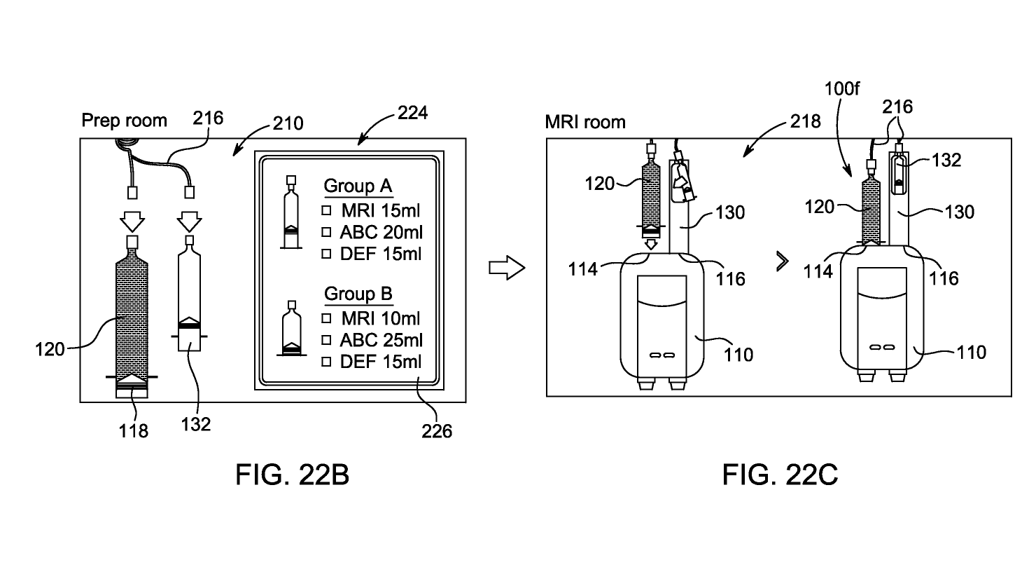

With reference to FIGS. 22A-22C, a system 100f for manual syringe identification is illustrated. The system 100f includes an injector 110, a saline syringe 120, and an adapter 130 containing a contrast syringe 132. The system 100f further includes an electronic device 224 including a visual display 226, allowing an operator to select the type of syringe being used from a list of available options. The electronic device 224 may be a dedicated electronic device, personal computer (PC), tablet PC, or smartphone including software and a user interface for selecting the type of syringe being used. The electronic device 224 may be integrated with or connected to other controls systems that control settings for the injector 110. As in previous embodiments, the operator prepares the saline syringe 120 and contrast syringe 132 in the prep room 210. Once the syringes 120, 132 are prepared, the operator manually enters information about the syringe 132 using the electronic device 224. The system 100f confirms that the syringe 132 and fluid contained therein are correct for the procedure being performed. Once the syringe 132 is identified and confirmed, the syringes 120, 132, adapter 130, and administration tubing 216 are transported to the MRI room 218 and loaded to the injector 110. The operator then begins the injection process, as described in the preceding embodiments.

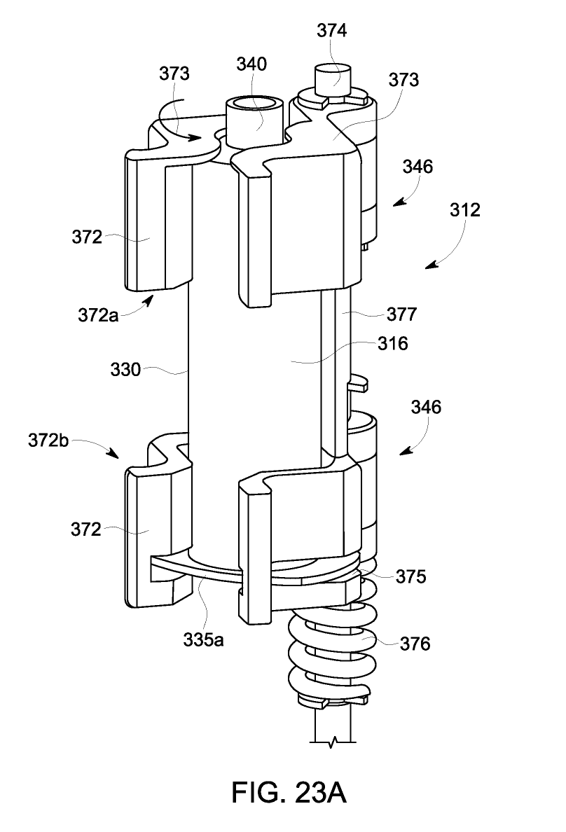

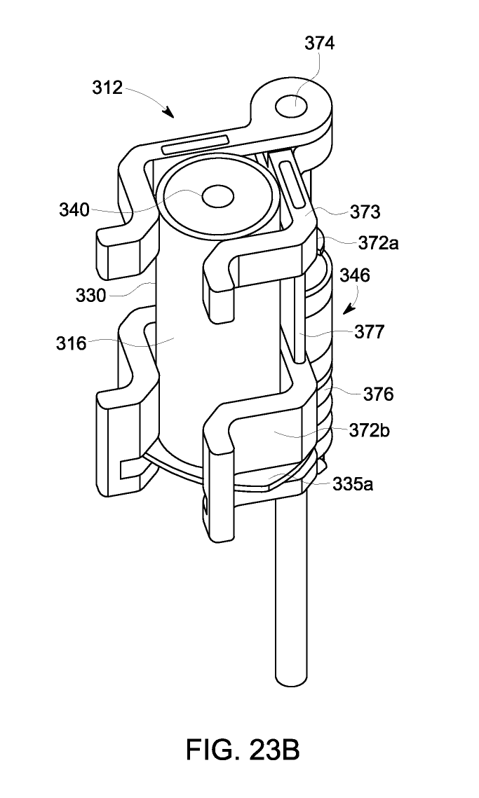

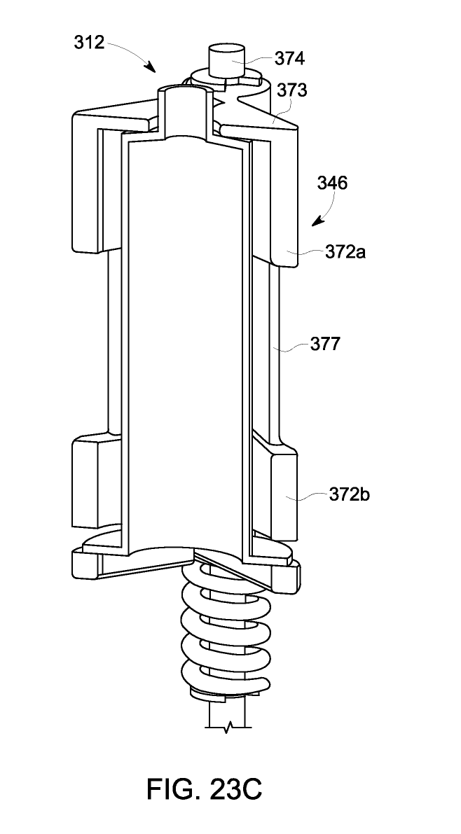

With reference to FIGS. 23A-23C, a further embodiment of an adapter 312 having radial or side supports 346 for holding a syringe 316 in correct alignment is illustrated. The adapter 312 includes two pairs of opposing v-shaped blocks 372 defining notches for receiving a portion of the syringe barrel, namely an upper pair 372a of v-shaped blocks and a lower pair 372b of v-shaped blocks.

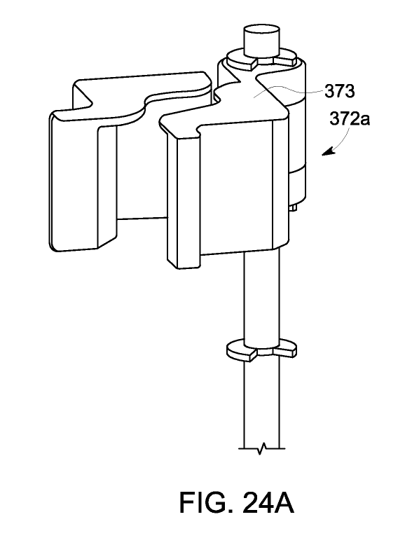

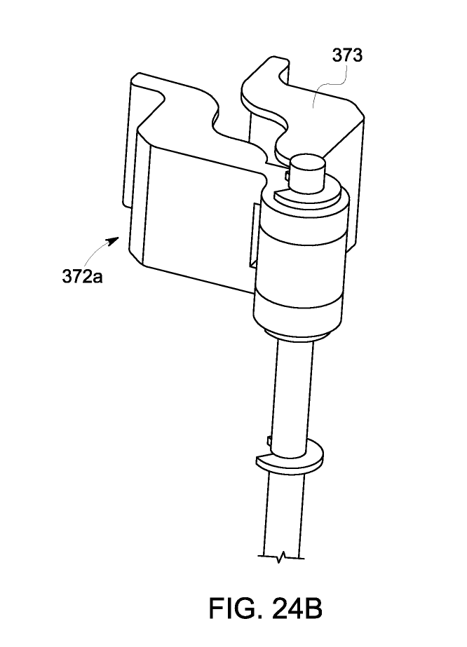

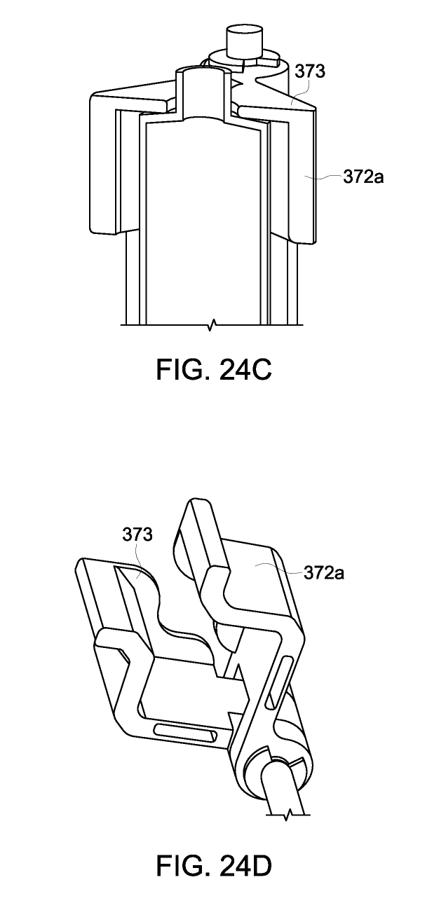

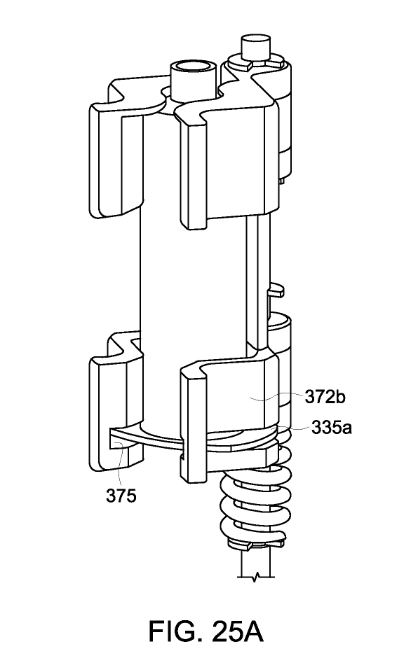

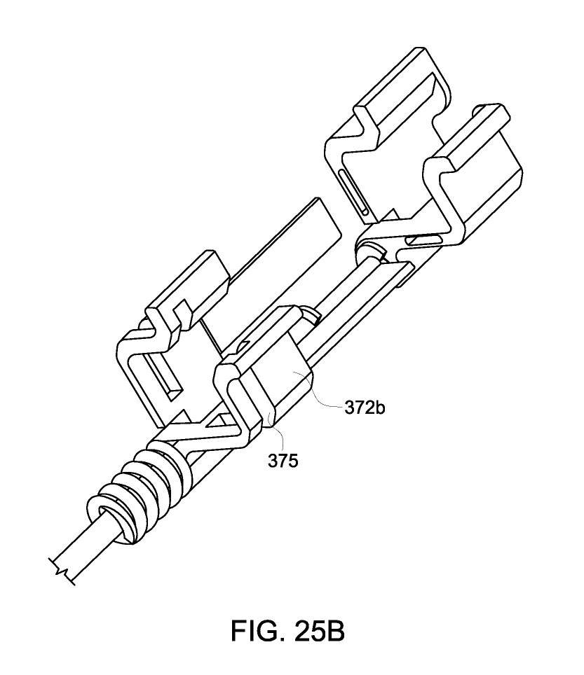

The upper pair 372a, which is illustrated in FIGS. 24A-24D, includes a flange 373 located at the top of the v-shaped blocks 372 for locating a nozzle 340 of the syringe 316. The lower pair 372b of v-shaped blocks, which are illustrated in FIGS. 25A and 25B, includes a slot 375 for receiving a drip flange 335a of the syringe 316. With continued reference to FIGS. 23A-23C, each pair 372a, 372b of v-shaped blocks is attached to a shaft or hinge 374 allowing the blocks 372 to pivot radially outward to receive the syringe 316. The upper pair 372a of v-shaped blocks is connected to the top (e.g., distal end) of the hinge 374 and prevented from moving axially by one or more support rings attached to the hinge 374. The lower pair 372b of blocks is capable of sliding along the hinge 374 in the axial direction, so that syringes 316 of different lengths can be inserted in the adapter 312. A snap ring may be positioned along the hinge 374 to limit travel of the lower pair 372b of blocks in the distal direction. The hinge 374 may be connected to a torsion spring 376 to bias the v-shaped blocks 372 to a closed position surrounding at least a portion of the syringe barrel 330. The upper pair 372a and lower pair 372b of v-shaped blocks may also be connected together via a tongue and groove attachment mechanism 377, in which a tongue extending from the lower pair 372b of blocks is inserted in a groove in the upper pair 372a of blocks. The tongue and groove mechanism 377 ensures that the v-shaped blocks 372 open and close in conjunction with one another. Accordingly, the likelihood that the syringe 316 will be inserted into the blocks 372 in an upright orientation is effectively increased.

While several embodiments of the universal adapter and syringe identification system are shown in the accompanying figures and described hereinabove in detail, other embodiments will be apparent to, and readily made by, those skilled in the art without departing from the scope and spirit of the disclosure. For example, it is to be understood that this disclosure contemplates that, to the extent possible, one or more features of any embodiment can be combined with one or more features of any other embodiment. Accordingly, the foregoing description is intended to be illustrative rather than restrictive.

What is claimed is:

1. A universal adapter for connecting a syringe to an injector comprising:
a body having a proximal end configured to connect to an injector;
at least one radial support connected to the body and biased in an inward direction, the at least one radial support defining a notch positioned to contact and engage a portion of a barrel of a syringe; and
at least one axial support biased to position the syringe in an axial direction toward a distal end of the adapter.

2. The universal adapter of claim 1, wherein the radial support and the axial support are configured to receive syringes across a range of different dimensions and geometries.

3. The universal adapter of claim 1, wherein the at least one radial support is configured to align a longitudinal axis of the syringe with a longitudinal axis of the body.

4. The universal adapter of claim 1, further comprising a plunger rod enclosed in a proximal portion of the body, the plunger rod being moveable in a distal direction to engage a plunger of the syringe.

5. The universal adapter of claim 1, wherein the at least one radial support comprises a block, and wherein the block is pivotally connected to a portion of the body, such that the block rotates about the portion of the body in a first direction when the syringe is being inserted into the adapter, and is biased to rotate about the portion of the body in a second direction, opposite the first direction, to engage the barrel of the syringe.

6. The universal adapter of claim 5, wherein the block comprises an outwardly flared surface, distinct from the notch, positioned such that contacting the outwardly flared surface causes the block to rotate about the portion of the housing in the first direction.

7. The universal adapter of claim 1, wherein the at least one radial support comprises a first block and a second block, wherein the first block and the second block are pivotally mounted to a portion of the body, and wherein the first block is biased about the portion of the body in a first direction and the second block is biased about the portion of the body in a second direction, the first direction being opposite the second direction.

8. The universal adapter of claim 1, wherein the syringe comprises a plunger rod extending in a proximal direction from a plunger, the plunger being slidably inserted in the syringe barrel.

9. The universal adapter of claim 8, further comprising a drive assembly moveable within the adapter and configured to engage a portion of the plunger rod of the syringe and to advance the plunger rod in a distal direction.

10. The universal adapter of claim 9, wherein the drive assembly comprises a plurality of finger members pivotally connected to a portion of the drive assembly, and biased in an inward direction to engage the portion of the plunger rod.

11. The universal adapter of claim 1, further comprising an injector interface configured to engage a portion of a linear actuator of the injector, the interface comprising a plurality of legs pivotally mounted to a portion of the interface and inwardly biased to grasp the portion of the linear actuator.

12. The universal adapter of claim 1, further comprising a forward load plate covering a distal opening of the adapter body, the load plate comprising a central opening configured to receive a distal end of the syringe.

13. The universal adapter of claim 12, wherein the forward load plate comprises an annular or partially annular riser surrounding the central opening, the riser comprising a tapered surface extending from an interior of the cavity toward a distal surface of the load plate.

14. The universal adapter of claim 1, wherein the axial support comprises at least one block defining a radial or latitudinal slot extending through at least a portion of the block for receiving a drip flange of the syringe.

15. A fluid delivery system comprising:
a syringe for delivering a fluid to a patient, the syringe comprising a barrel and a plunger slidably disposed within the barrel;
an injector comprising a linear actuator; and
a universal adapter configured to receive the syringe and to align the syringe with the linear actuator of the injector, such that the linear actuator can advance the plunger through the barrel to expel fluid from the syringe, the universal adapter comprising:
a body having a proximal end configured to connect to the injector,
at least one radial support connected to the body and biased in an inward direction, the at least one radial support defining a notch positioned to contact and engage the syringe barrel, and
at least one axial support biased to position the syringe in an axial direction toward a distal end of the adapter.

16. The universal adapter of claim 15, wherein the radial support and the axial support are configured to receive syringes across a range of different dimensions and geometries.

17. The fluid delivery system of claim 15, wherein the syringe comprises a plunger rod extending in a proximal direction from the plunger.

18. The fluid delivery system of claim 17, wherein the universal adapter further comprises a drive assembly moveable within the adapter housing and configured to engage a portion of the plunger rod to advance the plunger rod in a distal direction.

19. The fluid delivery system of claim 15, further comprising an injector interface configured to engage a portion of the linear actuator of the injector, the interface comprising a plurality of legs pivotally mounted to a portion of the interface and inwardly biased to grasp the portion of the linear actuator.

20. A syringe identification system comprising:
at least one syringe containing a medical fluid for injection to a patient;
an injector comprising a linear actuator configured to expel fluid from the syringe;
a universal adapter for receiving the syringe and for aligning the syringe with the linear actuator of the injector, the universal adapter comprising
a body having a proximal end configured to connect to the injector,
at least one radial support connected to the body and biased in an inward direction, the at least one radial support defining a notch positioned to contact and engage a portion of a barrel of the syringe, and
at least one axial support biased to position the syringe in an axial direction toward a distal end of the adapter; and
one or more sensors disposed on or associated with the universal adapter or the injector, the sensors being configured to obtain measurements for dimensions and geometries of the syringe,
wherein the measurements obtained by the one or more sensors are used to identify a type of syringe, a syringe fluid volume, syringe fluid flow characteristics, or any combination thereof.

21. The syringe identification system of claim 20, further comprising:
an identification tag disposed on the at least one syringe including or associated with identifying information about the syringe; and
a detector for determining the identifying information by reading the identification tag.

22. The syringe identification system of claim 21, wherein the identification tag comprises a one-dimensional bar code, a two-dimensional bar code, or a near-field communication device.
